(19) United States
(12) Patent Application Publication
(10) Pub. No.: US 2004/0235759 A1
(43) Pub. Date: Nov. 25, 2004

(54) Use of azalide antibiotic compositions for treating or preventing a bacterial or protozoal infection in mammals

(75) Inventors: Peter Connor Canning, Terre Haute, IN (US); Wayne Alan Boettner, Noank, CT (US)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 10/745,748

(22) Filed: Dec. 23, 2003

Related U.S. Application Data
(63) Continuation of application No. 09/829,672, filed on Apr. 10, 2001, now abandoned.
(60) Provisional application No. 60/199,961, filed on Apr. 27, 2000.

(51) Int. Cl. A61K 31/7052

(52) U.S. Cl. 514/28

(57) ABSTRACT

Methods for treating or preventing bacterial or protozoal infections in mammals by administering a single dose of an antibiotic composition comprising a mixture of azalide isomers and a pharmaceutically acceptable vehicle are disclosed. Methods for increasing acute or chronic injection-site toleration in mammals by administering a single dose of antibiotic compositions comprising a mixture of azalide isomers and a pharmaceutically acceptable vehicle are also disclosed. A combination comprising: an antibiotic composition comprising a mixture of azalide isomers, a pharmaceutically acceptable carrier, and instructions for use in a single-dose administration is also disclosed.

### BACKGROUND OF THE INVENTION

This invention relates to methods for using a pharmaceutical composition comprising a mixture of isomers of an azalide antibiotic compound for treating or preventing a bacterial or protozoal infection in mammals. The invention further relates to methods for increasing acute or chronic injection-site toleration in a mammal comprising administering a mixture of isomers of an azalide antibiotic. The invention also relates to a combination comprising a mixture of isomers of an azalide antibiotic, a pharmaceutically acceptable vehicle and instructions for use in a single-dose administration.

Macrolide antibiotic agents active against a wide variety of bacterial and protozoal infections in mammals, fish and birds have been previously reported (see, e.g., International Patent Publications WO 98/56802 and WO 99/12552). These compounds generally have a macrocyclic lactone ring of 12 to 22 carbon atoms to which one or more sugar moieties are attached. Macrolide antibiotics act on the 50S ribosomal subunit to inhibit protein synthesis in microorganisms. Examples of macrolide antibiotics include lincomycin, azithromycin, which is a derivative of erythromycin A, and other azalide compounds.

Development of pharmaceutical compositions containing azalide compounds as the active ingredient has presented significant challenges. Some azalides are capable of isomerizing in solution. Consequently, the production of a reproducible antibiotic composition comprising a single isomer or a fixed ratio of isomers has been difficult. Second, a composition containing a fixed amount of a particular azalide isomer may change over time. Third, the lactone ring and sugars of azalides are easily hydrolyzed in even mildly acidic or basic pH environments, decreasing the potency and shelf-life of an antibiotic composition.

It is an object of the present invention to provide methods for treating or preventing a bacterial or protozoal infection in mammals using antibiotic compositions that overcome the above-mentioned disadvantages.

Livestock animals that are stressed by a change in environment or diet, or by being housed with new animals carrying unfamiliar pathogens, are particularly susceptible to disease. The stress usually occurs when the animals are first sold, and therefore, these animals are known to be at risk. Many diseases are highly contagious and may cause high mortality and morbidity rates in a herd. Because most antibiotics tend to have short lifetimes in vivo, multiple doses are often required for disease prevention. In addition, sick animals require repeated doses of these drugs.

Accordingly, it is an object of the present invention to provide methods for treating or preventing a bacterial or protozoal infection in mammals comprising administering a single dose of a mixture of isomers of an azalide antibiotic compound.

Citation of any reference herein shall not be construed as indicating that such reference is prior art to the present invention.

### SUMMARY OF THE INVENTION

In a first embodiment, the present invention relates to a method for treating or preventing a bacterial or protozoal infection in a mammal, comprising administering to a mammal in need of such treatment-or prevention a single dose of an effective amount of a composition comprising: (a) a mixture of a compound of formula (I):

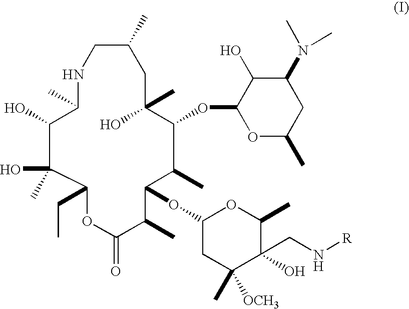

or a pharmaceutically acceptable salt thereof, and

a compound of formula (II):

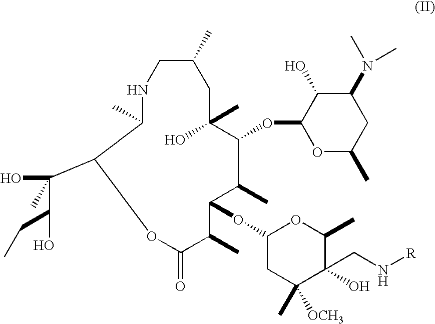

or a pharmaceutically acceptable salt thereof, wherein both R groups are identical and are selected from the group consisting of hydrogen, a C₁-C₁₀ straight or branched chain alkyl group, and a C₃-C₇ cycloalkyl group; and (b) a pharmaceutically acceptable vehicle.

In a second embodiment, the present invention relates to a method for increasing acute or chronic injection-site toleration in a mammal, comprising administering to a mammal in need thereof a single dose of an effective amount of a composition comprising: (a) a mixture of a compound of formula (I), or a pharmaceutically acceptable salt thereof, and a compound of formula (II), or a pharmaceutically acceptable salt thereof; and (b) a pharmaceutically acceptable vehicle.

In a third embodiment, the present invention relates to a combination comprising: (a) (1) a mixture of a compound of formula (I), or a pharmaceutically acceptable salt thereof and a compound of formula (II), or a pharmaceutically acceptable salt thereof; and (2) a pharmaceutically acceptable vehicle; and (b) instructions for use in a single-dose administration.

The present invention may be understood more fully by reference to the detailed description and illustrative examples which are intended to exemplify non-limiting embodiments of the invention.

### DETAILED DESCRIPTION OF THE INVENTION

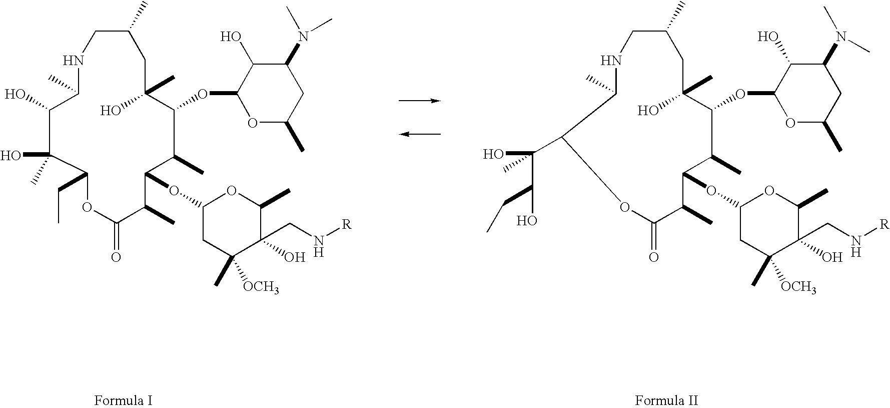

The present invention relates to methods for treating or preventing a bacterial or protozoal infection in mammals comprising administering a single dose of an effective amount of a pharmaceutical composition comprising a mixture of a compound of formula I, or a pharmaceutically acceptable salt thereof, and a compound of formula II, or a pharmaceutically acceptable salt thereof, wherein R is defined above; and a pharmaceutically acceptable vehicle. Preferably, R is n-propyl.

The compounds of formula I, which are 15-membered azalides, are isomeric with respect to the compounds of formula II, which are 13-membered azalides. Accordingly, as used herein, the term “mixture of isomers” refers to a mixture of a compound of formula I, or a pharmaceutically acceptable salt thereof, and its corresponding 13-membered azalide isomer, which is a compound of formula II, or a pharmaceutically acceptable salt thereof. In a preferred embodiment, the mixture of isomers comprises a compound of formula I and a compound of formula II in a ratio of about 90%±4% to about 10%±4%, respectively. The chemical name of the compound of formula I wherein R is n-propyl (the “N-(n-propyl) isomer I”) is (2R,3S,4R,5R,8R,10R,11R,12S,13S,14R)-13-((2,6-dideoxy-3-O-methyl-3-O-methyl-4-C-((propylamino)-methyl)-α-L-ribo-hexopyranosyl)oxy-2-ethyl-3,4,10-trihydroxy-3,5,8,10,12,14-hexamethyl-11-((3,4,6-trideoxy-3-(dimethylamino)-β-D-xylo-hexopyranosyl)oxy)-1-oxa-6-azacyclopentadecan-15-one. The chemical name of the compound of formula II wherein R is n-propyl (the “N-(n-propyl) isomer II”) is (3R,6R,8R,9R,10S,11S,12R)-11-((2,6-dideoxy-3-C-methyl-3-O-methyl-4-C-((propylamino)methyl-α-L-ribo-hexopyranosyl)oxy)-2-((1R,2R)-1,2-dihydroxy-1-methylbutyl)-8-hydroxy-3,6,8,10,12-pentamethyl-9-((3,4,6-trideoxy-3-(dimethylamino)-β-D-xylo-hexopyranosyl)oxy)-1-oxa-4-azacyclotridecan-13-one. A compound of formula I can be formed from a translactonization reaction of a compound of formula II. Likewise, a compound of formula II can be formed from a translactonization reaction of a compound of formula I.

Methods for obtaining compounds of formula I wherein R is H or C₁-C₁₀ alkyl are disclosed in International publication no. WO 98/56802, incorporated herein by reference. The methods disclosed in International publication no. WO 98/56802 can also be used to obtain compounds of formula I wherein R is C₃-C₇ cycloalkyl, particularly by selecting a desired C₃-C₇ cycloalkylamine in place of a C₁-C₁₀ alkylamine or an ammonia equivalent. Methods for obtaining compounds of formula II are described herein. The compounds of formula I and formula II are active antibiotic agents.

A composition comprising a compound of formula I and a compound of formula II in a ratio of about 90%±4% to about 10%±4% can be obtained rapidly using the methods disclosed herein independent of their starting ratio. It is believed that the about 90%±4% to about 10%±4% ratio of a compound of formula I, or a pharmaceutically acceptable salt thereof, and a compound of formula II, or a pharmaceutically acceptable salt thereof, constitutes an equilibrium mixture of isomers. Accordingly, the term “equilibrium mixture of isomers” as used herein refers to the mixture of isomers, wherein a compound of formula I, or a pharmaceutically acceptable salt thereof, and a compound of formula II, or a pharmaceutically acceptable salt thereof, are in a ratio of about 90%±4% to about 10%±4%, respectively. An antibiotic composition comprising the equilibrium mixture of isomers can be consistently produced and provides a standard for testing or consumer use. Thus, a composition comprising the equilibrium mixture of isomers is highly desirable.

Examples of C₁-C₁₀ straight or branched chain alkyl groups include, but are not limited to, methyl, ethyl, 1-propyl, 2-propyl, 1-butyl, 2-butyl, 2-methyl-1-propyl, 2-methyl-2-propyl, 1-pentyl, 2-pentyl, 3-pentyl, 2-methyl-1-butyl, 3-methyl-1-butyl, 2-methyl-3-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, 1-heptyl, 2-heptyl, 3-heptyl, 2-methyl-1-hexyl, 3-methyl-1-hexyl, 4-methyl-1-hexyl, 2-methyl-2-hexyl, 3-methyl-2-hexyl, 4-methyl-2-hexyl, 2,2-dimethyl-1-pentyl, 3,3-dimethyl-1-pentyl, 4,4-dimethyl-1-pentyl, 1-octyl, 2-octyl, 3-octyl, 4-octyl, 2-methyl-1-heptyl, 3-methyl-1-heptyl, 4-methyl-1-heptyl, 2-methyl-2-heptyl, 2,2-dimethyl-1-hexyl, 2-ethyl-1-hexyl, 3-ethyl-1-hexyl, 4-ethyl-1-hexyl, 1-nonyl, 2-nonyl, 3-nonyl, 4-nonyl, 2-methyl-1-octyl, 3-methyl-1-octyl, 4-methyl-1-octyl, 5-methyl-1-octyl, 2,2-dimethyl-1-heptyl, 2-ethyl-1-heptyl, 3-ethyl-1-heptyl, 4-ethyl-1-heptyl, 1-decyl, 2-methyl-1-nonyl, 3-methyl-1-nonyl, 4-methyl-1-nonyl, 5-methyl-1-nonyl, 2,2-dimethyl-1-octyl, 2-ethyl-1-octyl, 3-ethyl-1-octyl, 4-ethyl-1-octyl, and 5-ethyl-1-octyl.

Examples of C₃-C₇ cycloalkyl groups include, but are not llimited to, cyclopropyl, cyclobutyl, cyclopentyl, cyclohexyl, and cycloheptyl groups.

The phrase “pharmaceutically acceptable salt(s),” as used herein includes but are not limited to salts of basic amino groups that are present in compounds used in the present compositions. Compounds useful in the methods of the present invention, which are basic in nature, are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to acetic acid, benzenesulfonic acid, citric acid, hydrobromic acid, hydrochloric acid, D- and L-lactic acid, methanesulfonic acid, phosphoric acid, succinic acid, sulfuric acid, D- and L-tartaric acid, p-toluenesulfonic acid, adipic acid, aspartic acid, camphorsulfonic acid, 1,2-ethanedisulfonic acid, laurylsulfuric acid, glucoheptonic acid, gluconic acid, 3-hydroxy-2-naphthoic acid, 1-hydroxy-2-naphthoic acid, 2-hydroxyethanesulfonic acid, malic acid, mucic acid, nitric acid, naphthalenesulfonic acid, palmitic acid, D-glucaric acid, stearic acid, maleic acid, malonic acid, fumaric acid, benzoic acid, cholic acid, ethanesulfonic acid, glucuronic acid, glutamic acid, hippuric acid, lactobionic acid, lysinic acid, mandelic acid, napadisylic acid, nicotinic acid, polygalacturonic acid, salicylic acid, sulfosalicylic acid, tryptophanic acid, and mixtures thereof. The inorganic acids among the above are preferably used in the form of their aqueous solutions; more preferably, the inorganic acids are used in the form of their dilute, e.g., <2M, aqueous solutions. The organic acids among the above can be used in the form of dilute aqueous or organic solutions, wherein the organic solution comprises a solvent that sufficiently solvates both the organic acid and the compound of formula I.

Compounds useful in the methods of the present invention may form pharmaceutically acceptable salts with various amino acids, in addition to the acids mentioned above.

A compound of formula II can be obtained by contacting a compound of formula I with an acid or a base.

Acids useful in this regard include, but are not limited to inorganic acids, such as hydrochloric, hydrobromic, hydroiodic, hydrofluoric, sulfuric and nitric acids; and organic acids, such as formic, acetic, trifluoroacetic, methanesulfonic, trifluoromethanesulfonic, benzenesulfonic and p-toluenesulfonic acids. The inorganic acids are preferably used in the form of their aqueous solutions; more preferably, the inorganic acids are used in the form of their dilute, e.g., <2M, aqueous solutions. The organic acids can be used in the form of dilute aqueous or organic solutions, wherein the organic solution comprises a solvent that sufficiently solvates both the organic acid and the compound of formulae I and II.

Bases useful in this regard include inorganic bases, such as hydroxides of sodium, lithium, potassium, magnesium or calcium; carbonates and bicarbonates of sodium, lithium or potassium; and carbonates of magnesium or calcium bicarbonate or carbonate. Also useful are organic bases, such as triethylamine, ethyldiisopropylamine, pyridine, 4-dimethylaminopyridine, collidine, lutidine, and mixtures thereof. Preferably, the inorganic bases are used in the form of dilute aqueous solutions. Preferably the organic bases are used in the form of dilute organic solutions. Inorganic or organic bases are preferred over inorganic or organic acids.

The compounds of formula I can be added to the acid or base, or vice versa. Either way, the reaction of the compounds of formula I with the acid or base is facilitated by heating a mixture of a compound of formula I and an acid or base at a temperature of about room temperature to about 100° C., preferably at a temperature of about room temperature to about 60° C., and more preferably at a temperature of about 30° C. to about 40° C. Such heating can occur for a period of about 20 minutes to about 48 h, preferably for a period of about 1 hour to about 36 h.

A compound of formula II, or a pharmaceutically acceptable salt thereof, can also be obtained by heating a compound of formula I in the presence of solvent.

Such heating is achieved at a temperature of about room temperature to about 100° C., preferably at a temperature of about room temperature to about 60° C., and more preferably at a temperature of about 30° C. to about 40° C. The heating can occur for a period of about 20 minutes to about 48 h, preferably for a period of about 1 h to about 36 h.

Useful solvents are those that sufficiently solvate the compounds of formula I, and include, but are not limited to, lower alkanols, diethyl ether, acetone, acetonitrile, tetrahydrofuran, ethyl acetate, benzene, toluene, chloroform, metheylene chloride, dimethylformamide, dimethylsulfoxide, N-methylpyrrolidinone, and the like, and mixtures thereof.

However, the conversion of compounds of formula I to compounds of formula II proceeds most rapidly in a solvent system that comprises a protic solvent. Useful protic solvents include, but are not limited to, lower alkanols, such as methanol, ethanol, n-propanol, iso-propanol, n-butanol, iso-butanol and sec-butanol; phenolic compounds, such as phenol, halophenols, naphthols and the like; water; and mixtures thereof. It is to be pointed out, however, that the protic solvent is not a carboxylic acid.

Where the solvent system comprises a protic solvent, the protic solvent is present in an amount of about 10% to about 75% by volume, preferably in an amount of about 25% to about 60% by volume.

It will be understood by those skilled in the art that the protic solvent will be miscible in the solvent in which the compound of formula I is heated, when heated at the heating temperature.

Preferably, the solvent system comprises acetonitrile. More preferably, the solvent system further comprises a lower alkanol or water. Where the solvent system comprises a lower alkanol, the lower alkanol is preferably methanol.

The compounds of formula II can be isolated or purified via standard means, e.g., recrystallization; chromatography using a column, preparative plate or CHROMATOTRON® device; or by other means know to those skilled in the art. Where chromatography is employed to isolate or purify the compounds of formula II, the present inventors have discovered that an eluent system that comprises a hydrocarbon solvent and an organic amine provides enhanced separation results, relative to other eluent systems. Hydrocarbon solvents useful in this regard include, but are not limited to, pentane, hexane or hexanes, heptane, petroleum ether, benzene, toluene, xylenes, and the like. Preferably, the hydrocarbon solvent is hexane or hexanes. Useful organic amines include, but are not limited to, diethylamine, triethylamine, ethyldiisopropylamine, pyridine, 4-dimethylaminopyridine, collidine, lutidine, and mixtures thereof. Preferably, the organic amine is diethylamine.

Advantageously, the eluent system that comprises a hydrocarbon solvent and an organic amine further comprises a polar organic solvent. The addition of the polar organic solvent to the eluent system provides a better separation of the compounds of formula II from other compounds, relative to an eluent system that does not comprise a polar organic solvent. Useful polar organic solvents include, but are not limited to, lower alkanols, acetonitrile, dimethylformamide, dimethylsulfoxide, N-methylpyrrolidinone, 1,4-dioxane, tetrahydrofuran, diethyl ether, ethyl acetate, and the like. Preferably, the polar organic solvent is acetonitrile. More preferably, the eluent system comprises hexanes, diethylamine and acetonitrile.

The proportions of hydrocarbon solvent, organic amine, and optionally polar organic solvent can vary, but generally, the ratio of hydrocarbon solvent to organic amine will range from about 10:1 to about 1:1, preferably about 7:1 to about 2:1. Where the eluent system further comprises a polar organic solvent, the eluent system will contain the polar organic solvent at between about 1% to about 15% by volume, preferably at between about 1.5% to about 10% by volume.

The pharmaceutical compositions useful for the methods of the present invention comprise a mixture of isomers together with a suitable amount of a pharmaceutically acceptable vehicle so as to provide the form for proper administration to a mammal.

In a specific embodiment, the term “pharmaceutically acceptable” means approved by a regulatory agency of the Federal or a state government or listed in the U.S. Pharmacopeia or other generally recognized pharmacopeia for use in mammals. The term “vehicle” refers to a diluent, adjuvant, excipient, or carrier with which the mixture of isomers is administered. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents may be used. When administered to a mammal, the compositions of the invention and pharmaceutically acceptable vehicles are preferably sterile. Water is a preferred vehicle when the compositions of the invention are administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents. In a preferred embodiment, the pharmaceutically acceptable vehicle comprises: (i) water; (ii) one or more acids present at a total concentration of from about 0.2 mmol to about 1.0 mmol per mL of the mixture; and (iii) one or more water-miscible co-solvents present in an amount of from about 250 to about 750 mg per mL of the composition.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable vehicle is a capsule (see e.g., U.S. Pat. No. 5,698,155). Other examples of suitable pharmaceutical vehicles are described in “Remington's Pharmaceutical Sciences” by E. W. Martin.

Compositions comprising an equilibrium mixture of isomers, which are useful in the present invention, can be prepared as follows. The equilibrium mixture of isomers is obtained from a solution of the mixture isomers. Preferably, the mixture of isomers, when used to prepare the equilibrium mixture of isomers, comprises a compound of formula I that is substantially pure. By “substantially pure”, as used herein, unless otherwise indicated, is meant having a purity of at least 97%. In general, an equilibrium mixture of isomers is generated by heating a water solution of the mixture of isomers in the presence of one or more acids. In a preferred embodiment, a water solution of the mixture of isomers and one or more acids is heated to a temperature of between about 50° C. to about 90° C., preferably about 60° C. to about 80° C., for about 0.5 to about 24 hours, preferably about 1 to about 10 hours, at a pH of about 5.0 to about 8.0, preferably about 6.0 to about 8.0. Most preferably, a solution of the mixture of isomers is heated to a temperature of between about 65° C. to about 75° for about 1 to about 8 hours at a pH of about 6.5 to about 7.5 in the presence of one or more acids. The concentration of the mixture of isomers to be equilibrated can vary from about 50 mg/mL to about 500 mg/mL, more preferably from about 100 mg/mL to about 300 mg/mL, and most preferably from about 225 mg/mL to about 275 mg/mL of solution.

Suitable acids useful for obtaining the equilibrium mixture of isomers include, but are not limited to, acetic acid, benzenesulfonic acid, citric acid, hydrobromic acid, hydrochloric acid, D- and L-lactic acid, methanesulfonic acid, phosphoric acid, succinic acid, sulfuric acid, D- and L-tartaric acid, p-toluenesulfonic acid, adipic acid, aspartic acid, camphorsulfonic acid, 1,2-ethanedisulfonic acid, laurylsulfuric acid, glucoheptonic acid, gluconic acid, 3-hydroxy-2-naphthoic acid, 1-hydroxy-2-naphthoic acid, 2-hydroxyethanesulfonic acid, malic acid, mucic acid, nitric acid, naphthalenesulfonic acid, palmitic acid, D-glucaric acid, stearic acid, maleic acid, malonic acid, fumaric acid, benzoic acid, cholic acid, ethanesulfonic acid, glucuronic acid, glutamic acid, hippuric acid, lactobionic acid, lysinic acid, mandelic acid, napadisylic acid, nicotinic acid, polygalacturonic acid, salicylic acid, sulfosalicylic acid, tryptophanic acid, and mixtures thereof. Preferably, the one or more acids are citric and hydrochloric acid. When present, citric acid is present at a concentration of from about 0.02 mmol to about 0.3 mmol per mL of solution. In one embodiment, an acid concentration of from about 0.2 mmol to about 1.0 mmol per mL of solution is used. Without being bound by any theory, it is believed that the salt formed from the addition of an acid to a solution of the mixture of isomers exerts a buffering effect, because the azalide isomers themselves act as a base. Those of skill in the art will recognize that the amount of acid required for a desired pH will vary according to which acid is used, and that, in order to maintain a pH within the desired range, additional acid and/or a base may be added to the solution of acid and the mixture of isomers. Suitable bases include, but are not limited to, alkali metal hydroxides and carbonates, alkali metal bicarbonates, and alkaline earth hydroxides and carbonates. Sodium hydroxide and potassium hydroxide are preferred. The acids and bases described above are conveniently used in the form of their aqueous solutions.

Compositions comprising a mixture of isomers are useful for treating or preventing a bacterial or protozoal infection in a mammal. The compositions are also useful as intermediates for the formation of stabilized compositions and of stabilized, equilibrated compositions.

Methods for making stabilized compositions, which are useful in the methods of the present invention, comprising diluting the mixture of isomers with one or more water-miscible organic solvents (“co-solvent”). Methods for making stabilized, equilibrated compositions, which are also useful in the methods of the present invention, comprise diluting the equilibrated mixture of isomers with one or more co-solvents. The co-solvent does not significantly affect the ratio of a compound of formula I and a compound of formula II in the compositions, and in fact preserves their structural integrity. “Preserving the structural integrity” of a compound of formula I or a compound of formula II as used herein, includes, but is not limited to, retarding their rate of hydrolysis to, for example, descladinose azalide, and retarding their rate of byproduct formation of, for example, a formaldehyde and an acetaldehyde insertion product, defined below. Without being bound by any theory, it is believed that dilution with co-solvent improves the stability of the mixture of the mixture of isomers. Moreover, by virtue of the presence of co-solvent, any pain experienced upon injection of the stabilized compositions or the stabilized, equilibrated compositions may be less than that experienced from injection of a composition not so stabilized. Co-solvents useful for stabilizing the compositions include, but are not limited to, alcohols such as ethanol and isopropanol; glycol ethers such as diethylene glycol monomethyl ether, diethylene glycol butyl ether, diethylene glycol monoethyl ether and diethylene glycol dibutyl ether; polyethylene glycols such as polyethylene glycol-300 and polyethylene glycol-400; glycols such as propylene glycol and glycerine; pyrrolidones such as 2-pyrrolidone and N-methyl 2-pyrrolidone; glycerol formal; dimethyl sulfoxide; dibutyl sebecate; polyoxyethylene sorbitan esters such as polysorbate 80; and mixtures thereof. Preferably, co-solvents useful for stabilizing the compositions in injectable solutions include, but are not limited to, ethanol, polyethylene glycols such as polyethylene glycol-300 and polyethylene glycol-400, glycols such as propylene glycol and glycerine, pyrrolidones such as 2-pyrrolidone and N-methyl 2-pyrrolidone, glycerol formal, dimethyl sulfoxide, polyoxyethylene sorbitan esters such as polysorbate 80, and mixtures thereof, more preferably, glycerol formal, N-methyl 2-pyrrolidone and propylene glycol, and most preferably, propylene glycol. In one embodiment, co-solvent in an amount of about 250 to about 750 mg per mL of the pharmaceutical compositions is used to stabilize them. In a preferred embodiment, about 400 to about 600 mg of co-solvent per mL of the pharmaceutical compositions is used.

In a most preferred embodiment, about 450 to about 550 mg of co-solvent per mL of the pharmaceutical compositions is used.

In one embodiment, one or more co-solvents are added to the mixture of isomers prior to equilibration. In this case, the resulting mixture is heated to a temperature of between about 50° C. to about 90° C., preferably about 60° C. to about 80° C., for about 0.5 to about 24 hours, preferably for about 1 to about 10 hours, at a pH of about 5.0 to about 8.0, preferably at a pH of about 6.0 to about 8.0. In a preferred embodiment, equilibration of the mixture of isomers is carried out in the absence of co-solvent, which is added to the equilibrated compositions after they have cooled to about room temperature.

After addition of the co-solvent, the pH of the resulting solution can be re-adjusted to further improve stability of the composition. The pH is adjusted by methods known to those skilled in the art, such as for example by adding an amount of acid or base described above, e.g., as a 10% (w/w) stock solution, and measuring the pH of the resulting solution using, e.g., a pH meter. In one embodiment, the pH of the resulting solution, if necessary, is adjusted to about 4.5 to about 7.5, preferably about 5.0 to about 6.0, most preferably, about 5.2 to about 5.6.

Pharmaceutical compositions comprising a mixture of isomers and a pharmaceutically acceptable vehicle are useful in the methods of the present invention. Preferably, the pharmaceutical compositions further comprise water, one or more acids, and one or more water-miscible co-solvents. The amount of the mixture of isomers in the pharmaceutical compositions ranges from about 50 mg per mL of pharmaceutical composition to about 200 mg per mL of pharmaceutical composition. Preferably, the pharmaceutical compositions comprise from about 75 mg to about 150 mg, more preferably, from about 90 to about 110 mg, of the mixture of isomers per mL of pharmaceutical composition.

The pharmaceutical compositions can still further comprise one or more antioxidants. Antioxidants retard the rate of or prevent oxidative breakdown of the pharmaceutical compositions. Suitable antioxidants include, but are not limited to, sodium bisulfite, sodium sulfite, sodium metabisulfite, sodium thiosulfate, sodium formaldehyde sulfoxylate, 1-ascorbic acid, erythorbic acid, acetylcysteine, cysteine, monothioglycerol, thioglycollic acid, thiolactic acid, thiourea, dithiothreitol, dithioerythreitol, glutathione, ascorbyl palmitate, butylated hydroxyanisole, butylated hydroxytoluene, nordihydroguaiaretic acid, propyl gallate, α-tocopherol, and mixtures thereof. Those of skill in the art will recognize that the amount of antioxidant will vary according to which antioxidant is used. In a preferred embodiment, the antioxidant, when present, is present in an amount of from about 0.01 mg to about 10 mg per mL of pharmaceutical composition. In a more preferred embodiment, the antioxidant is monothioglycerol and present in an amount of from about 1 mg to about 8 mg per mL of pharmaceutical composition. In a most preferred embodiment, the antioxidant is monothioglycerol and present in an amount of from about 4 mg to about 6 mg per mL of pharmaceutical composition.

The pharmaceutical compositions optionally comprise one or more preservatives. Preservatives are useful for retarding the rate of or preventing proliferation of microorganisms, particularly when the pharmaceutical compositions are exposed to air. Useful preservatives are: effective against a broad spectrum of microorganisms; physically, chemically and microbiologically stable over the lifetime of the pharmaceutical compositions; non-toxic; adequately soluble; compatible with other components of the composition; and acceptable with respect to taste and odor. Suitable preservatives include, but are not limited to, benzalkonium chloride, benzethonium chloride, benzoic acid, benzyl alcohol, methylparaben, ethylparaben, propylparaben, butylparaben, sodium benzoate, phenol, and mixtures thereof. In a preferred embodiment, the one or more preservatives are selected from the group consisting of benzyl alcohol, methylparaben, propylparaben, a methylparaben/propylparaben combination, and phenol. When present, the one or more preservatives are present in an amount of from about 0.01 to about 10 mg per mL of the pharmaceutical compositions. Preferably, the one or more preservatives is phenol and present in an amount of from about 2.0 to about 5.0 mg per mL, more preferably, from about 2.0 to about 3.0 mg per mL, of the pharmaceutical compositions. One of skill in the art will recognize that the amount of preservative to be used in the present compositions will depend on which preservative is chosen, and that some preservatives may be used at lower concentrations, even lower than about 0.01 mg per mL of the pharmaceutical compositions.

In one embodiment, the pharmaceutical compositions useful in the methods of the invention have a pH of from about 5.0 to about 7.0 and comprise: (1) a mixture of isomers present in an amount of from about 50 mg to about 200 mg per mL of the pharmaceutical composition; (2) citric acid present in a concentration of from about 0.02 mmol to about 0.3 mmol per mL of the pharmaceutical composition and, optionally, an amount of hydrochloric acid effective to achieve the pH range; (3) propylene glycol, present in an amount of from about 250 to about 750 mg per mL of the pharmaceutical composition; (4) monothioglycerol, present in an amount of from about 1 mg to about 15 mg per mL of the pharmaceutical composition; and (5) water, present in an amount of from about 100 to about 750 mg per mL of the pharmaceutical composition. In a preferred embodiment, the mixture of isomers is an equilibrium mixture of isomers. In a more preferred embodiment, the equilibrium mixture of isomers is that wherein R is n-propyl.

In a preferred embodiment, the pharmaceutical compositions useful in the methods of the invention have a pH of from about 5.0 to about 6.0 and comprise: (1) a mixture of isomers present in an amount of from about 75 mg to about 150 mg per mL of the pharmaceutical composition; (2) citric acid present in an amount of from about 0.05 mmol to about 0.15 mmol per mL of the pharmaceutical composition and, optionally, an amount of hydrochloric acid effective to achieve the pH range; (3) propylene glycol, present in an amount of from about 400 to about 600 mg per mL of the pharmaceutical composition; (4) monothioglycerol, present in an amount of from about 1 mg to about 8 mg per mL of the pharmaceutical composition; and (5) water, present in an amount of from about 250 to about 550 mg per mL of the pharmaceutical composition. More preferably, the mixture of isomers is an equilibrium mixture of isomers. In a more preferred embodiment, the equilibrium mixture of isomers is that wherein R is n-propyl.

In a more preferred embodiment, the pharmaceutical compositions useful in the methods of the invention have a pH of from about 5.2 to about 5.6 and comprise: (1) a mixture of isomers present in an amount of from about 90 mg to about 110 mg per mL of the pharmaceutical composition; (2) citric acid present in an amount of from about 0.075 mmol to about 0.125 mmol per mL of the pharmaceutical composition, and an amount of hydrochloric acid effective to achieve the pH range; (3) propylene glycol, present in an amount of from about 450 to about 550 mg per mL of the pharmaceutical composition; (4) monothioglycerol, present in an amount of from about 4 mg to about 6 mg per mL of the pharmaceutical composition; and (5) water, present in an amount of from about 300 to about 500 mg per mL of the pharmaceutical composition. Most preferably, the mixture of isomers is an equilibrium mixture of isomers. In a more preferred embodiment, the equilibrium mixture of isomers is that wherein R is n-propyl.

Optionally, the pharmaceutical compositions useful in the methods of the present invention can be supplied to an end user, e.g., a physician or a veterinarian, together with instructions for use in a single-dose administration. Accordingly, the present invention provides a combination comprising a composition of the invention and instructions for use in a single-dose administration.

The pharmaceutical compositions can be prepared as follows. Reagents are added in a stainless steel- or glass-lined jacketed vessel with optional nitrogen overlay. Water for Injection is added to the reaction vessel, and agitation is begun. Each additional component is added while the mixture is continuously agitated. Acid in a concentration of about 0.02 mmol to about 0.5 mmol per mL of water is added and allowed to dissolve. An aqueous solution of an acid, e.g., a 10% (w/w) aqueous solution of hydrochloric acid, is optionally added to adjust the pH to a desired range and the solution is mixed. At this point, the mixture of isomers is added to the water and acid mixture slowly and in small quantities to avoid clumping. Here, a compound of formula I can be added prior to adding a compound of formula II, a compound of formula II can be added prior to adding a compound of formula I, or a compound of formula I and a compound of formula II can be added together. The mixture of isomers is allowed to dissolve, and the pH of the resulting solution is measured. In one embodiment, the mixture of isomers is from about 50 mg to about 500 mg per mL, preferably from about 100 to about 300 mg per mL, and most preferably from about 225 to about 275 mg per mL, of the resulting solution. The solution is then heated to a temperature of about 70° C. 10° C. and is maintained at this temperature until an equilibrium mixture of isomers is obtained. Methods for determining that an equilibrium mixture of isomers has been obtained include gel chromatography, thin-layer chromatography, and high-performance liquid chromatography. Generally, using the conditions described herein, an equilibrium mixture of isomers is obtained in about 1 to about 8 hours. Once the equilibrium mixture of isomers is obtained, the resulting solution is cooled to about 25° C.±10° C. This solution can be used as a pharmaceutical composition. Preferably, co-solvent is added in an amount of from about 250 to about 750 mg per mL of the pharmaceutical composition. Antioxidant is optionally added in an amount of from about 0.01 mg to about 10 mg per mL of the pharmaceutical composition. If present, preservative is added in an amount of from about 0.01 to about 10 mg per mL of the pharmaceutical composition, and the pH is adjusted to about 5.0 to about 8.0, preferably to about 5.0 to about 6.0, by adding acid and/or base, for example, as a 10% (w/w) aqueous solution or in solid form. The resulting mixture is diluted to a desired volume. In one embodiment, the final concentration of the equilibrium mixture of isomers is about 50 mg to about 200 mg, preferably about 75 mg to about 150 mg, and most preferably about 90 mg to about 110 mg per mL of the resulting pharmaceutical composition.

The resulting compositions are preferably sterilized, for example, by passing the compositions through a pre-filter, e.g., a 5-10 micron filter and then through a 0.2 micron final sterilizing filter that has been previously sterilized. The sterilizing filter is sterilized by moist-heat autoclaving for 60 minutes at 121° C., and is tested for integrity using a pressure-hold method prior to sterilization and after product filtration. The sterile solution is added to suitable containers, e.g., glass vials, that are sterilized and depyrogenated at 250° C. for 240 minutes in a dry-heat tunnel. The container head-space is flushed with an inert gas, e.g., argon or preferably, nitrogen. The containers are capped with stoppers that are depyrogenated by washing and sterilized by moist-heat autoclaving for 60 minutes at 121° C. The containers are then over-sealed. Those skilled in the art will recognize that minor modifications to the above can be used to prepare sterile compositions.

The present invention relates to methods for treating or preventing a mammal, comprising administering to a mammal in need of such treatment a pharmaceutically effective amount of a pharmaceutical composition. The pharmaceutical compositions can be used to treat infections by gram-positive bacteria, gram-negative bacteria, protozoa, and mycoplasma, including, but not limited to, Actinobacillus pleuropneumonia, Pasteurella multocida, Pasteurella haemolytica, H. parasuis, B. bronchiseptica, S. choleraesuis, S. pilo, Moraxella bovis, H. somnus, M. bovis, Eimeria zuernii, Eimeria bovis, A. marginale, M. hyopneumoniae, Lawsonia intracellularis, and Staphylococcus, Salmonella, Chlamydia, Coccidia, Cryptosporidia, E. coli, Haemophilus, Neospora, and Streptococcus species.

The term “treating” an infection, as used herein, unless otherwise indicated, means lessening the severity of or eradicating a bacterial infection or protozoal infection as provided in the methods of the present invention. By “preventing” an infection, as used herein, unless otherwise indicated, is meant preventing the establishment and deleterious proliferation of the one or more bacteria or protozoa in the body of a mammal.

As used herein, unless otherwise indicated, the terms “bacterial infection(s)” and “protozoal infection(s)” include bacterial infections and protozoal infections that occur in mammals, fish and birds as well as disorders associated with bacterial infections and protozoal infections that may be treated or prevented by administering antibiotics such as the compounds of the present invention. Such bacterial infections and protozoal infections, and disorders associated with such infections, include the following: pneumonia, otitis media, sinusitus, bronchitis, tonsillitis, and mastoiditis associated with infection by Streptococcus pneumoniae, Haemophilus influenzae, Moraxella catarrhalis, Staphylococcus aureus, or Peptostreptococcus spp.; pharynigitis, rheumatic fever, and glomerulonephritis associated with infection by Streptococcus pyogenes, Groups C and G streptococci, Clostridium diptheriae, or Actinobacillus haemolyticum; respiratory tract infections associated with infection by Mycoplasma pneumoniae, Legionella pneumophila, Streptococcus pneumoniae, Haemophilus influenzae, or Chlamydia pneumoniae; uncomplicated skin and soft tissue infections, abscesses and osteomyelitis, and puerperal fever associated with infection by Staphylococcus aureus, coagulase-positive staphylococci (i.e., S. epidermidis, S. hemolyticus, etc.), Streptococcus pyogenes, Streptococcus agalactiae, Streptococcal groups C-F (minute-colony streptococci), viridans streptococci, Corynebacterium minutissimum, Clostridium spp., or Bartonella henselae; uncomplicated acute urinary tract infections associated with infection by Staphylococcus saprophyticus or Enterococcus spp.; urethritis and cervicitis; and sexually transmitted diseases associated with infection by Chlamydia trachomatis, Haemophilus ducreyi, Treponema pallidum, Ureaplasma urealyticum, or Neiserria gonorrheae; toxin diseases associated with infection by S. aureus (food poisoning and Toxic shock syndrome), or Groups A, B, and C streptococci; ulcers associated with infection by Helicobacter pylori; systemic febrile syndromes associated with infection by Borrelia recurrentis; Lyme disease associated with infection by Borrelia burgdorferi; conjunctivitis, keratitis, and dacrocystitis associated with infection by Chlamydia trachomatis, Neisseria gonorrhoeae, S. aureus, S. pneumoniae, S. pyogenes, H. influenzae, or Listeria spp.; disseminated Mycobacterium avium complex (MAC) disease associated with infection by Mycobacterium avium, or Mycobacterium intracellulare; gastroenteritis associated with infection by Campylobacter jejuni; intestinal protozoa associated with infection by Cryptosporidium spp.; odontogenic infection associated with infection by viridans streptococci; persistent cough associated with infection by Bordetella pertussis; gas gangrene associated with infection by Clostridium perfringens or Bacteroides spp.; and atherosclerosis associated with infection by Helicobacter pylori or Chlamydia pneumoniae. Bacterial infections and protozoal infections and disorders associated with such infections that may be treated or prevented in mammals include the following: bovine respiratory disease associated with infection by P. haemolytica, P. multocida, Mycoplasma bovis, H. somnus or Bordetella spp.; calf enteric disease associated with infection by E. coli or protozoa (i.e., coccidia, cryptosporidia, etc.); dairy cow mastitis associated with infection by Staph. aureus, Strep. uberis, Strep. agalactiae, Strep. dysgalactiae, Klebsiella spp., Corynebacterium, Enterococcus spp., or E. coli; swine respiratory disease associated with infection by A. pleuro., P. multocida, or Mycoplasma spp.; swine enteric disease associated with infection by E. coli, Lawsonia intracellularis, Salmonella, or Serpulina hyodyisinteriae; cow footrot associated with infection by Fusobacterium spp.; cow metritis associated with infection by E. coli; cow hairy warts associated with infection by Fusobacterium necrophorum or Bacteroides nodosus; bovine keratoconjunctivitis (pink-eye) associated with infection by Moraxella bovis; cow premature abortion associated with infection by protozoa (i.e. neospora); porcine ileitis; bovine coccidiosis; urinary tract infection in dogs and cats associated with infection by E. coli; skin and soft tissue infections in dogs and cats associated with infection by Staph. epidermidis, Staph. intermedius, coagulase neg. Staph. or P. multocida; dental or mouth infections in dogs and cats associated with infection by Alcaligenes spp., Bacteroides spp., Clostridium spp., Enterobacter spp., Eubacterium, Peptostreptococcus, Porphyromonas, or Prevotella; pyoderma in cats and dogs; pneumonia in cats and dogs; and infections of horses associated with Actinobacillus equi, Rodococcus equi, Streptococcus equi, and Streptococcus zooepidemicus. Other bacterial infections and protozoal infections and disorders associated with such infections that may be treated or prevented in accord with the method of the present invention are referred to in J. P. Sanford et al., “The Sanford Guide To Antimicrobial Therapy,” 26th Edition, (Antimicrobial Therapy, Inc., 1996).

The antibacterial and antiprotozoal activity of the mixture of isomers useful in the methods of the present invention against bacterial and protozoal pathogens is demonstrated by the mixtures ability to inhibit growth of defined strains of human or mammalian pathogens.

### Assay I

Assay I, described below, employs conventional methodology and interpretation criteria and is designed to provide direction for chemical modifications that may lead to compounds that circumvent defined mechanisms of macrolide resistance. In Assay I, a panel of bacterial strains is assembled to include a variety of target pathogenic species, including representatives of macrolide resistance mechanisms that have been characterized. Use of this panel enables the chemical structure/activity relationship to be determined with respect to potency, spectrum of activity, and structural elements or modifications that may be necessary to obviate resistance mechanisms. Bacterial pathogens that comprise the screening panel are shown in the table below. In many cases, both the macrolide-susceptible parent strain and the macrolide-resistant strain derived from it are available to provide a more accurate assessment of the compounds' ability to circumvent the resistance mechanism. Strains that contain the gene with the designation of ermA/ermB/ermC are resistant to macrolides, lincosamides, and streptogramin B antibiotics due to modifications (methylation) of 23S rRNA molecules by an Erm methylase, thereby generally prevent the binding of all three structural classes. Two types of macrolide efflux have been described; msrA encodes a component of an efflux system in staphylococci that prevents the entry of macrolides and streptogramins while mefA/E encodes a transmembrane protein that appears to efflux only macrolides. Inactivation of macrolide antibiotics can occur and can be mediated by either a phosphorylation of the 2′-hydroxyl (mph) or by cleavage of the macrocyclic lactone (esterase). The strains may be characterized using conventional polymerase chain reaction (PCR) technology and/or by sequencing the resistance determinant. The use of PCR technology in this application is described in J. Sutcliffe et al., “Detection Of Erythromycin-Resistant Determinants By PCR”, Antimicrobial Agents and Chemotherapy, 40(11), 2562-2566 (1996). The assay is performed in microtiter trays and interpreted according to Performance Standards for Antimicrobial Disk Susceptibility Tests—Sixth Edition: Approved Standard, published by The National Committee for Clinical Laboratory Standards (NCCLS) guidelines; the minimum inhibitory concentration (MIC) is used to compare strains. A mixture of isomers is initially dissolved in dimethylsulfoxide (DMSO) as a 40 mg/ml stock solution.

| Strain Designation | Macrolide Resistance Mechanism(s) |
|---|---|
| Staphylococcus aureus 1116 | susceptible parent |
| Staphylococcus aureus 1117 | ermB |
| Staphylococcus aureus 0052 | susceptible parent |
| Staphylococcus aureus 1120 | ermC |
| Staphylococcus aureus 1032 | MsrA, mph, esterase |
| Staphylococcus hemolyticus 1006 | msrA mph |
| Streptococcus pyogenes 0203 | susceptible parent |
| Streptococcus pyogenes 1079 | ermB |
| Streptococcus pyogenes 1062 | susceptible parent |
| Streptococcus pyogenes 1061 | ermB |
| Streptococcus pyogenes 1064 | ermB |
| Streptococcus agalactiae 1024 | susceptible parent |
| Streptococcus agalactiae 1023 | ermB |
| Streptococcus pneumoniae 1016 | susceptible |
| Streptococcus pneumoniae 1046 | ermB |
| Streptococcus pneumoniae 1095 | ermB |
| Streptococcus pneumoniae 1175 | mefE |
| Streptococcus pneumoniae 0085 | susceptible |
| Haemophilus influenzae 0131 | susceptible |
| Moraxella catarrhalis 0040 | susceptible |
| Moraxella catarrhalis 1055 | Erythromycin intermediate resistance |
| Escherichia coli 0266 | susceptible |

Assay II is utilized to test for activity against Pasteurella multocida and Assay III is utilized to test for activity against Pasteurella haemolytica.

### Assay II

This assay is based on the liquid dilution method in microliter format. A single colony of P. multocida (strain 59A067) is inoculated into 5 ml of brain heart infusion (BHI) broth. A solution is prepared by solubilizing 1 mg of a mixture of isomers in 125 μl of dimethylsulfoxide (DMSO). Dilutions of the mixture of isomers are prepared using uninoculated BHI broth. The concentrations of the mixture of isomers used range from 200 μg/ml to 0.098 μg/ml by two-fold serial dilutions. The P. multocida inoculated BHI is diluted with uninoculated BHI broth to make a 10⁴ cell suspension per 200 μl. The BHI cell suspensions are mixed with respective serial dilutions of the mixture of isomers, and incubated at 37° C. for 18 hours. The minimum inhibitory concentration (MIC) is equal to the concentration of the mixture exhibiting 100% inhibition of growth of P. multocida as determined by comparison with an uninoculated control.

### Assay III

This assay is based on the agar dilution method using a Steers Replicator. Two to five colonies isolated from an agar plate are inoculated into BHI broth and incubated overnight at 37° C. with shaking (200 rpm). The next morning, 300 μl of the fully grown P. haemolytica preculture is inoculated into 3 ml of fresh BHI broth and is incubated at 37° C. with shaking (200 rpm). The appropriate amounts of a mixture of isomers are dissolved in ethanol and a series of two-fold serial dilutions are prepared. Two ml of the respective serial dilution is mixed with 18 ml of molten BHI agar and solidified. When the inoculated P. haemolytica culture reaches 0.5 McFarland standard density, about 5 μl of the P. haemolytica culture is inoculated onto BHI agar plates containing the various concentrations of the mixture of isomers using a Steers Replicator and incubated for 18 hours at 37° C. Initial concentrations of the mixture range from 100-200 μg/ml. The MIC is equal to the concentration of the mixture exhibiting 100% inhibition of growth of P. haemolytica as determined by comparison with an uninoculated control.

Most preferably, the microdilution assay is performed using cation-adjusted Mueller-Hinton broth according to NCCLS guideline M31-A, Vol. 19, No. 11, “Performance standards for antimicrobial disk and dilution susceptibility tests for bacteria isolated from animals,” June 1999 (ISBN 1-56238-377-9), which is herein incorporated by reference. This assay may be used to determine the MIC of a compound against both P. haemolytica and P. multocida. For example, the equilibrium mixture of isomers was tested according to this standard, against P. haemolytica (ATCC 14003), and found to have a MIC of 1 μg/mL. When the equilibrium mixture of isomers was tested according to this standard, against P. multocida (ATCC 43137), the MIC was found to be 1 μg/mL.

### Assay IV

The in vivo activity of the pharmaceutical compositions of the present invention can be determined by conventional animal protection studies well known to those skilled in the art, usually carried out in mice.

Mice are allotted to cages (10 per cage) upon their arrival, and allowed to acclimate for a minimum of 48 hours before being used. Animals are inoculated with 0.5 ml of a 3×10³ CFU/ml bacterial suspension (P. multocida strain 59A006) intraperitoneally. Each experiment has at least 3 non-medicated control groups including one infected with 0.1× challenge dose and two infected with 1× challenge dose; a 10× challenge data group may also be used. Generally, all mice in a given study can be challenged within 30-90 minutes, especially if a repeating syringe (such as a Cornwall® syringe) is used to administer the challenge. Thirty minutes after challenging has begun, the first pharmaceutical composition treatment is given. It may be necessary for a second person to begin pharmaceutical composition dosing if all of the animals have not been challenged at the end of 30 minutes. The routes of administration are subcutaneous or oral doses. Subcutaneous doses are administered into the loose skin in the back of the neck whereas oral doses are given by means of a feeding needle. In both cases, a volume of 0.2 ml is used per mouse. Compositions are administered 30 minutes, 4 hours, and 24 hours after challenge. A control composition of known efficacy administered by the same route is included in each test. Animals are observed daily, and the number of survivors in each group is recorded. The P. multocida model monitoring continues for 96 hours (four days) post challenge.

The PD₅₀ is a calculated dose at which the pharmaceutical composition tested protects 50% of a group of mice from mortality due to the bacterial infection which would be lethal in the absence of treatment.

The pharmaceutical compositions useful for the methods of the present invention show antibacterial activity in one of the above-described assays, particularly in Assay IV.

The present invention further relates to methods for increasing acute or chronic injection-site toleration in a mammal, comprising administering to a mammal in need of such treatment a pharmaceutically effective amount of a composition comprising (a) a mixture of isomers and (b) a pharmaceutically acceptable vehicle. By “increasing acute injection-site toleration,” as used herein, is meant that when a composition of the invention is administered via injection, decreasing the amount of swelling and/or inflammation at the site of the injection, particularly that which is present at about 24 to 48 hours after injection, compared to the amount of swelling and/or inflammation present at about 24 to 48 hours after injection with antibiotic agents other than the mixture of isomers, such as, for example, MICOTIL. By “increasing chronic injection-site toleration,” as used herein, is meant decreasing the amount of tissue necrosis at the site of injection that is present about 2 weeks after injection, compared to the amount of tissue necrosis present 2 weeks after injection with antibiotic agents other than the mixture of isomers, such as, for example, MICOTIL. In a preferred embodiment, the mixture of isomers is an equilibrium mixture of isomers. In another embodiment, the mixture of isomers is that wherein R is n-propyl.

The pharmaceutical compositions useful for the methods of the invention can be used to treat humans, cattle, horses, sheep, swine, goats, rabbits, cats, dogs, and other mammals in need of such treatment. In particular, the pharmaceutical compositions useful for the methods of the invention can be used to treat, inter alia, bovine respiratory disease, swine respiratory disease, bovine infectious keratoconjunctivitis, bovine coccidiosis, porcine ileitis, bovine mastitis, bovine enteric disease, porcine enteric disease, canine pyoderma, feline pyoderma, canine pneumonia, feline pneumonia, canine soft-tissue diseases, feline soft-tissue diseases, pasteurellosis, anaplasmosis, and infectious keratinitis. The pharmaceutical compositions may be administered through oral, intramuscular, intravenous, subcutaneous, intra-ocular, parenteral, topical, intravaginal, or rectal routes. For administration to cattle, swine or other domestic mammals, the pharmaceutical compositions may be administered in feed or orally as a drench composition. Preferably, the pharmaceutical compositions are injected intramuscularly, intravenously or subcutaneously. In a preferred embodiment, the pharmaceutical compositions are administered in a single dose ranging from about 0.5 mg of the mixture of isomers per kg of body weight (mg/kg) to about 20 mg/kg. In a more preferred embodiment, the pharmaceutical compositions are administered in a single dose ranging from about 1.25 mg/kg to about 10 mg/kg. In a most preferred embodiment, the pharmaceutical compositions are administered in a single dose ranging from about 2.0 mg/kg to about 5.0 mg/kg. Most preferably, the pharmaceutical compositions are administered subcutaneously.

Anti-bacterial and/or anti-protozoal drugs other than the mixture of isomers can be co-administered with, administered prior to, or administered subsequent to administration of the present compositions, and multiple doses of those drugs may be useful. However, the present compositions are administered only once, i.e., in a single dose. By “single dose,” as used herein, is meant that a single administration of the pharmaceutical compositions is capable of treating or preventing a bacterial or protozoal infection. That is, while a subsequent dose of the pharmaceutical compositions may provide an added benefit, it is not required in the present methods. Furthermore, when administered in a single dose, the compositions of the invention do not need to comprise a larger amount of the mixture of isomers than would be present when administered in multiple doses.

The methods of the present invention are based in part on Applicants surprising discovery that the mixture of isomers have a long half-life (about 28 hours) in the tissues and peripheral circulation. Those of skill in the art will readily recognize that variations in dosages can occur depending upon the species, weight and condition of the subject being treated, its individual response to the pharmaceutical compositions, and the particular route of administration chosen. In some instances, dosage levels below the lower limit of the aforesaid ranges may be therapeutically effective, while in other cases still larger doses may be employed without causing any harmful side effects, provided that such larger doses are first divided into several small doses for administration throughout the day.

The following Examples further illustrate methods of obtaining compositions useful for the methods of the present invention. It is to be understood that the present invention is not limited to the specific details of the Examples provided below.

### EXAMPLE 1

Synthesis of N-(n-propyl) isomer II. To a 2 L erlenmeyer flask was added desmethylazithromycin (190.5 g, 259.2 mmol), methylene chloride (572 mL), and magnesium sulfate (38 g). The mixture was stirred for 10 minutes then filtered into a 5 L round bottom flask. Additional methylene chloride (2285 mL) was added and the solution cooled to 0-5° C. CBZ-CI (58.4 mL) was then added over 10 minutes. The reaction stirred at −0° C. for 6 hrs then at ambient temperature overnight. HPLC analysis indicated the presence of residual starting material such that the reaction was re-cooled to ˜0° C. and additional CBZ-CI (19.5 mL) was added in a single portion. The reaction stirred for 5.5 hrs at 0° C. then for 2.5 hrs at ambient temperature. TLC indicated a complete reaction. The reaction was quenched with saturated aqueous sodium bicarbonate (953 mL) and the phases separated. The organic phase was dried over magnesium sulfate, then filtered and concentrated to afford the compound of formula (III):

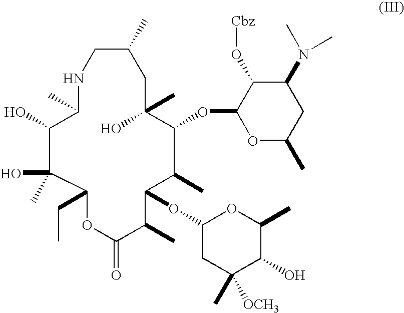

To a 5 L round bottom flask containing the compound of formula (III) (225.3 g) in methylene chloride (901 mL) and DMSO (450 mL) at −65° C. was added trifluoroacetic anhydride (82.4 mL). The temperature was maintained at ˜60° C. throughout the addition which was complete in 9 minutes. The reaction was stirred at −65 to −70° C. for 20 minutes. The reaction was quenched with triethylamine (145 mL) then stirred at −600 to −65° C. for 20 minutes. To the reaction mixture was then added water (1127 mL) over 3 minutes, at which point the temperature rose to −2° C. The reaction mixture was stirred for 10 minutes and the phases were allowed to separate. The organic phase was washed with water, (675 mL) then with saturated aqueous sodium chloride (675 mL). The organic phase was dried over magnesium sulfate then filtered and organic solvents removed by distillation. MTBE was added and distilled to remove all traces of methylene chloride and DMSO. Additional MTBE was added to a total volume of 3380 mL. Dibenzoyl-D-tartaric acid monohydrate (87.8 g) in MTBE (1126 mL) was added to form a thick slurry. The mixture was heated to reflux and stirred overnight. After cooling to ambient temperature, the solids were collected on a Buchner funnel and rinsed with MTBE. The solids were dried in a drying oven at 40° C. to afford 258.3 g of the dibenzoyl tartrate salt of the compound of formula (IV):

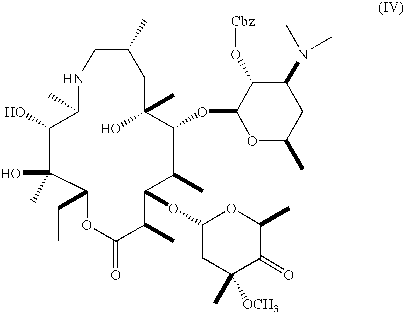

To a 3 L round bottom flask was added methylene chloride (800 mL) and the dibenzoyl tartrate salt of the compound of formula (IV) (188 g). Water (400 mL) and potassium carbonate (45.5 g) were added and the mixture stirred at ambient temperature for 5 minutes. The organic phase was separated, then washed with water (250 mL) and dried over magnesium sulfate. Drying agent was removed by filtration, and the resultant solution evaporated under a stream of nitrogen to a final volume of 623 mL to afford a free-base ketone.

To a 5 L round bottom flask was added THF (623 mL) and trimethylsulfonium bromide (74.7 g). The resultant slurry was cooled to −10° C. and potassium tert-butoxide (54.4 g) added. The reaction mixture was stirred for 10 minutes at −10° C. then cooled to −70° C. over 5 minutes. A solution of the free-base ketone was added over 11 minutes, keeping the temperature between −60 and −65° C. HPLC indicated the reaction was complete after 90 minutes. The reaction was quenched at −60° C. using a solution of ammonium chloride (315 g) in water (1800 mL). The temperature rose to −5° C. during the quench. The reaction mixture was warmed to 5-10° C., and the phases separated. The organic phase was dried over sodium sulfate then filtered and concentrated to afford the compound of formula (V), (117.4 g) as a yellow foam. HPLC indicated a purity of 61.4% by peak area.

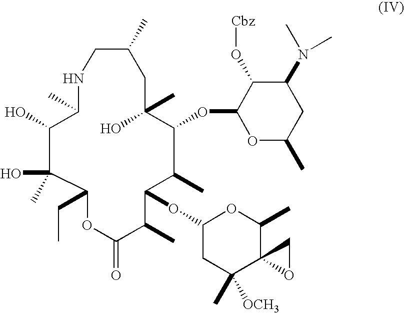

To a solution of the compound of formula (V) (275 g, 312 mmol) in dry methanol (2.75 L) was added potassium iodide (518 g, 3.12 mol) and n-propylamine (250 mL, 3.04 mol). The mixture was stirred overnight at 45° C. TLC indicated a complete reaction. The reaction was concentrated on a rotary evaporator and the residue partitioned between water (2.5 L) and methylene chloride (2.5 L). The pH of the aqueous phase was adjusted to 6.7 using 3N aqueous HCl. The extraction was repeated one additional time. Combined aqueous phases were combined with fresh methylene chloride (1.5 L) and the pH of the aqueous phase adjusted to 8.5 using solid potassium carbonate. The phases were separated and the aqueous phase re-extracted twice with additional methylene chloride. Combined organic phases were dried over sodium sulfate, then filtered. The filtrate was concentrated on a rotary evaporator to afford a beige foam (230 g). Purification of the foam was effected on a slurry-packed silica gel column using 19/3 (v/v) hexanes-diethylamine as the mobile phase. In this manner, 125 g of crude product afforded 72 g N-(n-propyl) isomer I as a white, amorphous foam.

N-(n-propyl) isomer I was dissolved in acetonitrile (0.5 L) at ambient temperature. Deionized water (1 L) was then added, which caused precipitation. Additional acetonitrile (0.5 L) was then added to afford a homogenous solution which was stirred at ambient temperature for 30 hrs. HPLC analysis indicated the formation of a new component that comprised 20% total peak area.

Organic solvent was removed on a rotary evaporator. Potassium carbonate (30 g) was added to the aqueous residue followed by methylene chloride (0.3 L). The mixture was shaken and the lower organic phase removed. Two additional extractions (2×0.3 L) were also performed. Combined organic phases were dried over sodium sulfate, then filtered and the resultant solution concentrated to a dry foam (−10 g).

The resultant mixture of N-(n-propyl) isomer I and N-(n-propyl) isomer II was dissolved in a mixture of methylene chloride and 19/3 (v/v) hexanes-diethylamine, and placed on a slurry-packed silica gel column, then eluted with the 19/3 system. The eluant was switched to 19/6 hexanes-diethylamine in fraction 56. Fraction 9-17 were combined and concentrated to a dry foam which contained only unreacted starting material. Fractions 52-72 were combined and concentrated, and contained N-(n-propyl) isomer II (79% purity by HPLC).

### EXAMPLE 2

Table 1 below shows the effect of pH, temperature, acid type, and concentration of N-(n-propyl) isomer I on the equilibration reaction rate and on levels of major impurities following equilibration. Replicated experiments (data not shown) demonstrated reproducibility of results. The equilibrium ratio of N-(n-propyl) isomer I and N-(n-propyl) isomer II (about 90%±4% to about 10%±4%, respectively) was consistent for all experiments. Analysis of the data indicates that pH and temperature have a significant effect on the time required for equilibration. Without being bound by any theory, lower equilibration temperatures or lower pH values generally result in substantially longer equilibration times. Equilibration time can also depend on, inter alia, the concentration of starting material, and the type and concentration of the acid used. N-(n-propyl) isomer I at a concentration of up to about 300 mg per mL of composition was heated to a temperature of about 40° C. to about 80° C. in the presence of one or more acids at a concentration of about 0.2 mmol to about 1.0 mmol per mL of mixture and with a sufficient quantity of hydrochloric acid to achieve a pH of about 6.5 to about 7.5 for up to about 20 hours to produce an equilibrium mixture of isomers that is about 95%-98% pure. Equilibration kinetic parameters and impurity levels for equilibration of N-(n-propyl) isomer I and N-(n-propyl) isomer II were determined as a function of pH, equilibration temperature, type of acid, and N-(n-propyl) isomer I concentration and are listed in Table 1. Known methods, including high performance liquid chromatography (“HPLC”), nuclear magnetic resonance spectroscopy (“NMR”), gas chromatography (“GC”), mass spectrometry (“MS”), liquid chromatography/mass spectrometry (“LC/MS”), GC/MS, and thin layer chromatography (“TLC”), can be used to identify the impurities. “DS” refers to N-(n-propyl) isomer I prior to equilibration and is included for comparison.

Equilibrium mixtures of isomers were prepared and assayed as follows. 40 mL of solution were prepared in each of experiments 1A-11A, and each solution was divided into 1 mL aliquots prior to heating in order to more easily monitor equilibration at different time points. 20 mL of solution were prepared in each of experiments 12B-24B, and each solution was divided into 0.7 mL aliquots prior to heating. 100 mL of solution were prepared in each of experiments 25C-28C, 200 mL of solution were prepared in each of experiments 29C-30C, and equlibration was monitored from 0.5 mL aliquots removed from the solutions. 60 mL of solution were prepared in each of experiments 31D-33D and 35D-41D, 170 mL of solution were prepared in experiment 34D, and equilibration was monitored from 0.5 mL aliquots removed from the solutions. From 7,200 mL to 54,000 mL of solution were prepared in each of experiments 42E-46E, and equilibration was monitored by removing from 2 mL to 5 mL aliquots from the solutions. From 35 mL to 50 mL of solution were prepared in each of experiments 47F-50G, and each solution was divided into 1 mL aliquots prior to heating. Water was added to the appropriate container, followed by the type and amount of acid listed in column 4 of Table 1. The term “qs” preceding the acid type refers an amount of the acid sufficient to achieve the pH listed in column 2. Where 0.1 M citric or tartaric acid was used, hydrochloric acid was also added in a quantity sufficient to obtain the pH listed in column 2. Where an acid concentration is recited in column 4 (e.g., “0.1 M citric”), this is the concentration of acid in a solution having an equilibrated mixture of N-(n-propyl) isomer I and N-(n-propyl) isomer II present in a concentration of 100 mg/ml. The mixture of water and acid was stirred until all of the acid was dissolved (about 5 minutes or less for smaller volumes, and about 20 minutes for larger volumes). N-(n-propyl) isomer I was added slowly and in small portions to avoid clumping, and the resulting mixture was stirred vigorously until dissolved (less than 30 minutes for smaller volumes, and about 60-120 minutes for larger volumes). After dissolution of N-(n-propyl) isomer 1, the pH of the resulting solution was measured. If the pH was lower than the pH listed in column 2, it was raised to the pH listed in column 2 with 10% sodium hydroxide. If the pH was higher than the pH listed in column 2, it was lowered with the appropriate acid(s). For each experiment, the solution was heated at the temperature noted in column 3 until an equilibrium mixture of N-(n-propyl) isomer I and N-(n-propyl) isomer II was obtained, as determined by one of the HPLC assays described below. In some experiments, mixtures were heated for a period of time longer than required for equilibration (percentages greater than 100% in column 8) to determine the effects of prolonged heat on the degree of impurity.

To monitor equilibration, reaction mixture aliquots were assayed by HPLC at various times during equilibration. For the majority of equilibration experiments shown in Table 1, aliquots were diluted with 40 mM potassium phosphate buffer (pH 6.0) to a concentration of approximately 0.5 mg of N-(n-propyl) isomer I and N-(n-propyl) isomer II per mL total sample volume and subjected to chromatography using an Asahipak ODP-50, 5 μm, 250×4.0 mm column (40% acetonitrile/35% methanol/25% 40 mM potassium phosphate; pH 8.5 mobile phase; flow rate 0.7 mL/minutes; room temperature) on an HP 1090 Liquid Chromatograph equipped with an external Applied Biosystems 783A Programmable Absorbance Detector. Peaks were detected by monitoring ultraviolet absorption at 210 nm. For the remaining equilibration experiments shown in Table 1 (experiments 31D-46E), aliquots were diluted with 20% acetonitrile/50% methanol/30% 50 mM potassium phosphate (pH 5.5) to a concentration of 1.0 mg of N-(n-propyl) isomer I and N-(n-propyl) isomer II per mL of total sample volume and subjected to chromatography using a YMC Pro-Pack C₁₈, 3 μm, 50×2.0 mm column (20% acetonitrile/50% methanol/30% 50 mM potassium phosphate; pH 7.0 mobile phase; flow rate 0.5 mL/minutes; room temperature) on an HP 1090 Liquid Chromatograph with internal UV Detector. Peaks were detected by monitoring ultraviolet absorption at 210 nm. Relative amounts of N-(n-propyl) isomer I and N-(n-propyl) isomer II were determined by taking the ratio of their relative chromatogram-peak areas. Under the above HPLC conditions, N-(n-propyl) isomer I has a retention time of approximately 13-23 minutes, and N-(n-propyl) isomer II has a relative retention time (“RRT”) of approximately 0.8 to 0.9. By “RRT” is meant a retention time relative to that of N-(n-propyl) isomer I under the above-described HPLC conditions.

The purity of equilibrated samples in Table 1 was determined using HPLC according to one of three procedures. In experiments 1A-24B, 48F, and 50G, aliquots were diluted with 25 mM potassium phosphate buffer (pH 5.5) to a concentration of 1.25 mg of N-(n-propyl) isomer I and N-(n-propyl) isomer II per mL total sample volume and assayed using an Eclipse XDB-C₈, 5 μm, 250×4.6 mm column (22% acetonitrile/58% methanol/20% 25 mM potassium phosphate; pH 8.0 mobile phase; flow rate 0.6 mL/minutes; room temperature) on a Waters Alliance 2690 Separation Module with BAS CC-5/LC-4C Amperometric Detector. Peaks were detected electrochemically with one electrode at +0.70 V, a second electrode at +0.88 V, and a range of 0.5 μA. In experiments 25C-41D, aliquots were diluted with 50 mM citric acid (pH 5.5) to a concentration of 0.25 mg of the mixture of N-(n-propyl) isomer I and N-(n-propyl) isomer II per mL of total sample volume and assayed using a YMC Pro-Pack C₁₈, 3 μm, 150×4.6 mm column (70% methanol/30% 50 mM phosphate; pH 7.0 mobile phase; flow rate 1 mL/minutes; room temperature) on the Waters Alliance. system. Peaks were detected electrochemically with only one electrode at +0.90 V. In experiments 42E-43E, aliquots were diluted with 50 mM citric acid (pH 5.5) to a concentration of 0.25 mg of the mixture of N-(n-propyl) isomer I and N-(n-propyl) isomer II per mL of total sample volume and assayed using a YMC Pro-Pack C₁₈, 3 μm, 150×4.6 mm column (70% methanol/30% 50 mM phosphate; pH 7.0 mobile phase; flow rate 1 mL/minute; room temperature) on an HP 1090 Liquid Chromatograph with BAS CC-5/LC-4C Amperometric Detector. Peaks were detected electrochemically with only one electrode at +0.90 V. The percentage of the equilibrium mixture of N-(n-propyl) isomer I and N-(n-propyl) isomer II (column 9) and impurities (column 10) relative to the assayed sample was determined using the areas under the peaks in the chromatograms. Some of the detected impurities were: a descladinose azalide (its RRT being approximately 0.26 on an Eclipse XDB-C₈ column), an acetaldehyde insertion product (its RRT being approximately 1.75 on an Eclipse XDB-C₈ column), and a formaldehyde insertion product (its RRT being approximately 1.6 on an Eclipse XDB-C₈ column).

The descladinose azalide has the structure:

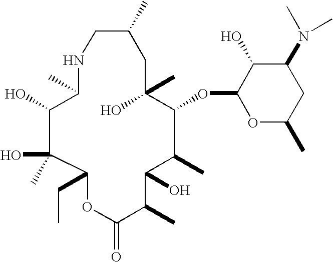

The acetaldehyde insertion product has the structure:

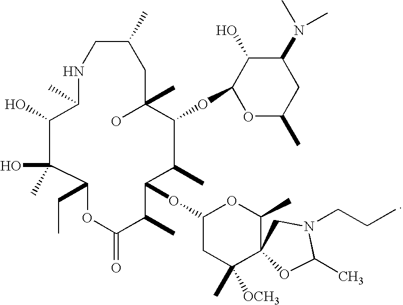

The formaldehyde insertion product has the structure:

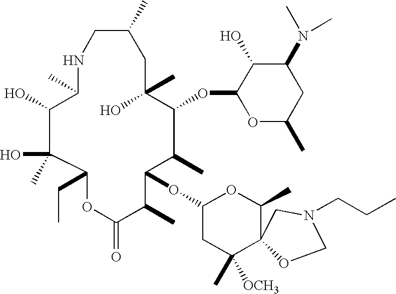

The descladinose azalide, the acetaldehyde insertion product, and the formaldehyde insertion product, and pharmaceutically acceptable salts thereof, have antibiotic properties and are useful as antibiotic agents.

The experiments of groups A and B (identified by the letter following the experiment number) in Table 1 were performed to determine the effects of pH, temperature, type of acid, concentration of acid, and N-(n-propyl) isomer I concentration on equilibration. The experiments of group C in Table 1 illustrate the effects of pH and temperature on equilibration. The experiments of group D in Table 1 illustrate the effects of pH, temperature, and acid concentration on equilibration. The experiments of group E in Table 1 illustrate a preferred method of equilibration, that is, at a pH of about 7.0, an equilibration temperature of about 70° C., and N-(n-propyl) isomer I concentration of about 250 mg/mL. Experiments in group F tested the effects of alternate acids and equilibration temperatures, and experiment G was performed in the presence of 50% propylene glycol co-solvent.

Results of these experiments indicate that, even under a variety of conditions, equilibration of the mixture of N-(n-propyl) isomer I and N-(n-propyl) isomer II consistently results in the formation of from about 90%±4% of N-(n-propyl) isomer I and about 10%±4% of N-(n-propyl) isomer II. Equilibration temperature and pH appear to have the largest effect on equilibration rate, with higher temperatures generally leading to faster rates, even with higher concentrations of N-(n-propyl) isomer 1. In most cases, however, longer equilibration times resulted in higher concentration of impurities, and therefore, optimal equilibration conditions are those leading to relatively high equilibration rates, i.e., that form the equilibrium mixture of N-(n-propyl) isomer I and N-(n-propyl) isomer II in 1-3 hours.

TABLE 1

| | | | | Initial | | | | | |
|---|---|---|---|---|---|---|---|---|---|
| | | | | N-(n-propyl) | % of N-(n- | | | % | |
| Experiment | | Equilibration | | isomer I | propyl) | time to | | Equilibrium | |
| Number & | | temperature | | concentration | isomer II at | equilibration | % of time to | mixture of | % |
| Group | pH | (°C) | acid | (mg/mL) | equilibration | (hrs) | equilibration | isomers | Impurities |
| DS | | | | | | | | 98.39 | 1.61 |
| 1A | 6.3 | 65 | qs citric | 112 | 12.2 | 5.1 | 100 | 96.09 | 3.91 |
| | | | | | | | 150 | 94.03 | 5.97 |
| 2A | 6.0 | 80 | qs phosphoric | 75 | 11.6 | 2 | 100 | 94.57 | 5.43 |
| | | | | | | | 370 | 86.71 | 13.29 |
| 3A | 7.0 | 70 | qs citric | 75 | 11.5 | 1.4 | 100 | 96.36 | 3.64 |
| | | | | | | | 640 | 89.62 | 10.38 |
| 4A | 7.0 | 50 | qs citric | 150 | 11.5 | 10.9 | 100 | 97.76 | 2.24 |
| | | | | | | | 200 | 96.94 | 3.06 |
| 5A | 6.0 | 60 | qs phosphoric | 150 | 11.2 | 12.9 | 100 | 96.52 | 3.48 |
| | | | | | | | 170 | 95.18 | 4.82 |
| 6A | 6.0 | 60 | qs citric | 75 | 12.3 | 17.6 | 100 | 96.15 | 3.85 |
| | | | | | | | 170 | 94.95 | 5.05 |
| 7A | 6.0 | 80 | qs citric | 150 | 11.9 | 2.2 | 100 | 96.15 | 3.85 |
| | | | | | | | 160 | 95.20 | 4.80 |
| 8A | 7.0 | 50 | qs phosphoric | 75 | 11.4 | 7.2 | 100 | 97.79 | 2.21 |
| | | | | | | | 150 | 96.47 | 3.53 |
| 9A | 7.0 | 70 | qs phosphoric | 150 | 12.1 | 1.2 | 100 | 97.66 | 2.34 |
| | | | | | | | 160 | 96.70 | 3.30 |
| 10A | 6.5 | 65 | qs citric | 112 | 11.3 | 3.3 | 100 | 96.63 | 3.37 |
| | | | | | | | 190 | 94.64 | 5.36 |
| 11A | 6.5 | 65 | qs citric | 112 | 12.4 | 3.6 | 100 | 96.87 | 3.13 |
| | | | | | | | 200 | 94.76 | 5.24 |
| 12B | 7.0 | 70 | qs citric | 150 | 12.5 | 1.4 | 100 | 94.33 | 5.67 |
| | | | | | | | 150 | 94.00 | 6.00 |
| 13B | 7.25 | 65 | 0.1 M citric/ | 225 | 11.2 | 3.3 | 100 | 94.55 | 5.45 |
| | | | qs HCl | | | | 150 | 94.26 | 5.74 |
| 14B | 7.25 | 65 | qs citric | 225 | 11.2 | 2.5 | 100 | 94.75 | 5.25 |
| | | | | | | | 150 | 94.17 | 5.83 |
| 15B | 7.0 | 70 | 0.1 M citric/ | 300 | 11.0 | 3.6 | 100 | 93.08 | 6.92 |
| | | | qs HCl | | | | 150 | 92.74 | 7.26 |
| 16B | 7.5 | 70 | 0.1 M citric/ | 150 | 11.6 | 1.4 | 100 | 94.98 | 5.02 |
| | | | qs HCl | | | | 150 | 94.82 | 5.18 |
| 17B | 7.0 | 60 | qs citric | 300 | 11.4 | 4.9 | 100 | 93.93 | 6.07 |
| | | | | | | | 150 | 93.80 | 6.20 |
| 18B | 7.5 | 60 | qs citric | 150 | 12.1 | 2.6 | 100 | 94.00 | 6.00 |
| | | | | | | | 150 | 93.89 | 6.11 |
| 19B | 7.5 | 60 | 0.1M citric/ | 300 | 11.3 | 4.5 | 100 | 93.89 | 6.11 |
| | | | qs HCl | | | | 150 | 93.78 | 6.22 |
| 20B | 7.5 | 70 | qs citric | 300 | 11.3 | 1.5 | 100 | 93.88 | 6.12 |
| | | | | | | | 150 | 93.65 | 6.35 |
| 21B | 7.0 | 60 | 0.1 M citric/ | 150 | 12.3 | 4.1 | 100 | 94.31 | 5.69 |
| | | | qs HCl | | | | 150 | 94.27 | 5.73 |
| 22B | 7.25 | 65 | 0.1 M citric/ | 225 | 11.7 | 3.0 | 100 | 94.51 | 5.49 |
| | | | qs HCl | | | | 150 | 94.24 | 5.76 |
| 23B | 7.25 | 65 | citric | 225 | 12.3 | 2.3 | 100 | 94.23 | 5.77 |
| | | | | | | | 150 | 94.10 | 5.90 |
| 24B | 7.0 | 70 | citric | 300 | 11.4 | 2.2 | 100 | 94.43 | 5.57 |
| 25C | 7.5 | 75 | 0.1 M citric/ | 250 | 11.8 | 1.3 | 100 | 98.59 | 1.41 |
| | | | qs HCl | | | | | | |
| 26C | 7.5 | 65 | 0.1 M citric/ | 250 | 10.5 | 3.0 | 100 | 98.78 | 1.22 |
| | | | qs HCl | | | | | | |
| 27C | 6.5 | 75 | 0.1 M citric/ | 250 | n/a | 4.0 | 85 | 98.74 | 1.26 |
| | | | qs HCl | | | | | | |
| 28C | 6.5 | 65 | 0.1 M citric/ | 250 | n/a | 9.9 | 50 | 98.91 | 1.09 |
| | | | qs HCl | | | | | | |
| 29C | 7.0 | 70 | 0.1 M citric/ | 250 | 11.1 | 1.8 | 100 | 98.90 | 1.10 |
| | | | qs HCl | | | | | | |
| 30C | 7.0 | 70 | 0.1 M citric/ | 250 | 11.3 | 2.2 | 100 | 98.91 | 1.09 |
| | | | qs HCl | | | | | | |
| 31D | 7.0 | 70 | 0.125 M citric/ | 250 | 10.2 | 2.6 | 100 | — | — |
| | | | qs HCl | | | | | | |
| 32D | 7.0 | 70 | 0.125 M citric/ | 250 | 10.1 | 2.8 | 100 | — | — |
| | | | qs HCl | | | | | | |
| 33D | 7.0 | 70 | 0.125 M citric/ | 250 | 10.2 | 2.6 | 100 | | |
| | | | qs HCl | | | | | | |
| 34D | 7.0 | 70 | 0.175 M citric/ | 250 | 10.5 | 2.5 | 100 | | |
| | | | qs HCl | | | | | | |
| 35D | 7.0 | 70 | 0.1 M citric/ | 250 | 10.6 | 2.2 | 100 | | |
| | | | qs HCl | | | | | | |
| 36D | 7.5 | 75 | 0.1 M citric/ | 250 | 11.2 | 1.0 | 100 | | |
| | | | qs HCl | | | | | | |
| 37D | 7.5 | 65 | 0.1 M citric/ | 250 | 11.1 | 1.9 | 100 | | |
| | | | qs HCl | | | | | | |
| 38D | 7.0 | 70 | 0.1 M citric/ | 250 | 10.8 | 2.4 | 100 | | |
| | | | qs HCl | | | | | | |
| 39D | 8.0 | 70 | 0.1 M citric/ | 250 | 11.1 | 1.1 | 100 | | |
| | | | qs HCl | | | | | | |
| 40D | 6.5 | 75 | 0.1 M citric/ | 250 | 10.6 | 1.9 | 100 | | |
| | | | qs HCl | | | | | | |
| 41D | 6.5 | 65 | 0.1 M citric/ | 250 | 10.8 | 5.8 | 100 | | |
| | | | qs HCl | | | | | | |
| 42E | 7.0 | 70 | 0.1 M citric/ | 250 | 11.6 | 1.1 | 100 | 96.00 | 4.00 |
| | | | qs HCl | | | | | | |
| 43E | 7.5 | 70 | 0.1 M tartaric/ | 250 | 12.0 | 2.0 | 100 | 94.90 | 5.10 |
| | | | qs HCl | | | | | | |
| 44E | 7.0 | 70 | 0.1 M | 250 | 13.6 | 1.4 | 100 | — | — |
| | | | citric/ | | | | | | |
| | | | qs HCl | | | | | | |
| 45E | 6.8 | 70 | 0.1 M | 250 | 12.7 | 1.1 | 100 | — | — |
| | | | citric/ | | | | | | |
| | | | qs HCl | | | | | | |
| 46E | 6.9 | 70 | 0.1 M | 250 | 12.4 | 1.3 | 100 | — | — |
| | | | citric/ | | | | | | |
| | | | qs HCl | | | | | | |
| 47F | 7 | 70 | qs citric | 250 | 10.8 | 1.4 | 100 | 94.78 | 5.22 |
| 48F | 7 | 70 | 0.1 M | 250 | 11.5 | 7.7 | 100 | — | — |
| | | | tartaric/ | | | | | | |
| | | | qs HCl | | | | | | |
| 49F | 7.4 | 39 | qs | 1 | 11.1 | 6.7 | 100 | — | — |
| | | | phosphoric | | | | | | |
| 50G | 7 | 70 | qs citric | 75 | 9.4 | 2.7 | 100 | — | — |

### EXAMPLE 3

The stability of compositions comprising an equilibrium mixture of N-(n-propyl) isomer I and N-(n-propyl) isomer II stored at 50° C. for 12 weeks and stabilized with co-solvent is shown in Table 2 below. The results indicate that the compositions containing no co-solvent are significantly less stable than compositions containing co-solvent in an amount of from about 250 to about 500 mg per mL of the composition (experiments 1A-11B). Compositions having a pH of about 5.4 and containing propylene glycol in an amount of from about 450 to about 550 mg per mL of the composition are the most stable. Other co-solvents may be used to stabilize the compositions (experiments 1E-2E); however, propylene glycol is preferred. As shown in Table 2, stability is dependent on pH, and it can also be dependent on type and quantity of acid used, and concentration of the equilibrated mixture of isomers.

These compositions were prepared as follows. After heating to the desired temperature (column 2) and allowing the mixture of water, acid, and N-(n-propyl) isomer I to equilibrate for the time shown in column 3, equilibrium mixtures of isomers were allowed to cool to room temperature. When the mixtures reached room temperature, the appropriate amount of the desired co-solvent was added (column 6). The percentage of co-solvent shown in column 6 is a weight-to-volume percentage (e.g., 50% PG is 500 mg propylene glycol per mL of pharmaceutical composition). If an antioxidant or a preservative was used, the appropriate amounts were added (columns 8 and 9). The pH of the solution was measured and adjusted to the value in column 5 by adding one or more acids and/or 10% w/w sodium hydroxide. The volumes of the resulting solutions were then adjusted by adding water. The compositions were filtered through a 0.2 micron sterilizing filter. Vials were filled in a laminar-flow hood, and the vial head space was flushed with the appropriate gas mixture (column 10) before sealing.

Equilibration and purity were monitored using HPLC as described above in Example 2. The stability of stabilized, equilibrated compositions sealed in glass vials was determined after storage for 12 weeks at 50° C. The effects of concentration of the equilibrium mixture of N-(n-propyl) isomer I and N-(n-propyl) isomer II, pH, co-solvent amount and type, type and concentration of acid, exposure to air, presence of preservatives, and presence of antioxidants were monitored. Results are shown in Table 2.

Experiments 1A-3A were performed to monitor the effect of equilibrium mixture concentration on stability. Experiments 2A, 6A, and 7A were performed to monitor the effect of pH on stability. Experiments 2A, 4A, and 5A show the effect of co-solvent amount on stability, and experiments 3A and 8A show the effect of using citric acid alone, as opposed to mixtures of citric and phosphoric acid, for obtaining an acidic pH. Experiments 1B-11B show the effects of pH and propylene glycol (“PG”) co-solvent on stability. Experiments 1C and 2C show the effect of using tartaric acid alone, as opposed to a mixture of tartaric and hydrochloric acid, for obtaining an acidic pH. Experiments 9B-11B and 3C show the effects of a preservative on stability of the mixture, and experiments 9B-11B, 4C, and 5C show the effects of an antioxidant on stability of the mixture. Experiments 6C and 7C show the effects of using a mixture of tartaric and hydrochloric acid or a mixture of citric and hydrochloric acid on stability. Experiments 1D-12D show the effects of different amounts of monothioglycerol (“MTG”) antioxidant and different degrees of oxygen exposure on stability. Experiments 4D-6D and 13D-18D demonstrate the effects of pH of the composition and acid concentration on stability.

Results of these experiments indicate that after storage for 12 weeks at 50° C., the equilibrated compositions that contain at least 50% propylene glycol and have a pH ranging from about 5.2 to about 5.5 retain greater than 93% of the initial concentration of the equilibrium mixture of N-(n-propyl) isomer I and N-(n-propyl) isomer II. The highest level of impurities was found in a composition having no co-solvent (experiment 4A). Accordingly, the presence of co-solvent surprisingly and unexpectedly limits the amount of impurities. High levels of impurities were found after 12 weeks in compositions having less than 40% co-solvent and a pH of less than 5.0. The concentration of the acid also affects stability of the pharmaceutical compositions. Compositions with relatively low concentrations of acid (about 20 mM) and a pH of about 5.4 show the greatest stability after storage. However, low acid concentrations result in low buffer strength, which leads to fluctuating pH and may lead to a relatively high degree of impurity under other time or temperature conditions.

TABLE 2

| | | | Concentration | | | | | | |
|---|---|---|---|---|---|---|---|---|---|
| | | | of equilibrium | | | | | | |
| | | | mixture of | | | | | | |
| | | | N-(n-propyl) | | | | | | |
| | | | isomer I and | | | | | | |
| | | | N-(n-propyl) | | | | | | |
| Experiment | Equil. | Equil. | isomer II in | | Co-solvent | | | | Anti- |
| number | temp | time | pharmaceutical | | type and | | Acid | Preser- | oxidant |
| and Group | (° C.) | (hrs) | comp. (mg/mL) | pH | amount | | concentration(M) | vative | (mg/mL) |
| 1A | 60 | 16 | 10 | .0 | 50% | PG | qs citric | — | — |
| 2A | 60 | 16 | 30 | .0 | 50% | PG | qs citric | — | — |
| 3A | 60 | 16 | 100 | .0 | 50% | PG | qs citric | — | — |
| 4A | 60 | 16 | 30 | .0 | — | PG | qs citric | — | — |
| 5A | 60 | 16 | 30 | .0 | 25% | PG | qs citric | — | — |
| 6A | 60 | 16 | 30 | .5 | 50% | PG | qs citric | — | — |
| 7A | 60 | 16 | 30 | .5 | 50% | PG | qs citric | — | — |
| 8A | 60 | 24 | 100 | .0 | 50% | PG | 0.23 M | — | — |
| | | | | | | | citric/ | | |
| | | | | | | | qs H₃PO₄ | | |
| 1B | 70 | 1.5 | 100 | .0 | 25% | PG | qs citric | — | — |
| 2B | 70 | 1.5 | 100 | .0 | 50% | PG | qs citric | — | — |
| 3B | 70 | 1.5 | 100 | .5 | 25% | PG | qs citric | — | — |
| 4B | 70 | 1.5 | 100 | .5 | 50% | PG | qs citric | — | — |
| 5B | 70 | 1.5 | 100 | .25 | 20% | PG | qs citric | — | — |
| 6B | 70 | 1.5 | 100 | .25 | 55% | PG | qs citric | — | — |
| 7B | 70 | 1.5 | 100 | .75 | 37.5% | PG | qs citric | — | — |
| 8B | 70 | 1.5 | 100 | .75 | 37.5% | PG | qs citric | — | — |
| 9B | 70 | 1.5 | 100 | .25 | 37.5% | PG | qs citric | — | — |
| 10B | 70 | 1.5 | 100 | .25 | 37.5% | PG | qs citric | — | — |
| 11B | 70 | 1.5 | 100 | .25 | 37.5% | PG | qs citric | — | — |
| 1C | 70 | 1.5 | 100 | 5.25 | 37.5% | PG | qs tartaric | — | — |
| 2C | 70 | 1.5 | 100 | 5.25 | 37.5% | PG | 0.1 M | — | — |
| | | | | | | | tartaric/ | | |
| | | | | | | | qs HCl | | |
| 3C | 70 | 1.5 | 100 | 5.25 | 37.5% | PG | qs citric | phenol | — |
| 4C | 70 | 1.5 | 100 | 5.25 | 37.5% | PG | qs citric | — | 5 |
| | | | | | | | | | MTG |
| 5C | 70 | 1.5 | 100 | 5.25 | 37.5% | PG | qs citric | — | 5 |
| | | | | | | | | | propyl |
| | | | | | | | | | gallate |
| 6C | 70 | 1.5 | 100 | 5.50 | 50% | PG | 0.1 M | — | 5 |
| | | | | | | | tartaric/ | | MTG |
| | | | | | | | qs HCl | | |
| 7C | 70 | 1.5 | 100 | 5.50 | 50% | PG | 0.1 M | — | 5 |
| | | | | | | | citric/ | | MTG |
| | | | | | | | qs HCl | | |
| 1D | 70 | 1.5 | 100 | 5.40 | 50% | PG | 0.1 M | — | 10 |
| | | | | | | | citric/ | | MTG |
| | | | | | | | qs HCl | | |
| 2D | 70 | 1.5 | 100 | 5.40 | 50% | PG | 0.1 M | — | 10 |
| | | | | | | | citric/ | | MTG |
| | | | | | | | qs HCl | | |
| 3D | 70 | 1.5 | 100 | 5.40 | 50% | PG | 0.1 M | — | 5 |
| | | | | | | | citric/ | | MTG |
| | | | | | | | qs HCl | | |
| 5D | 70 | 1.5 | 100 | 5.40 | 50% | PG | 0.1 M | — | 5 |
| | | | | | | | citric/ | | MTG |
| | | | | | | | qs HCl | | |
| 6D | 70 | 1.5 | 100 | 5.40 | 50% | PG | 0.1 M | — | 5 |
| | | | | | | | citric/ | | MTG |
| | | | | | | | qs HCl | | |
| 7D | 70 | 1.5 | 100 | .40 | 50% | PG | 0.1 M | — | 5 |
| | | | | | | | citric/ | | mM |
| | | | | | | | qs HCl | | MTG |
| 8D | 70 | 1.5 | 100 | .40 | 50% | PG | 0.1 M | — | 5 |
| | | | | | | | citric/ | | MTG |
| | | | | | | | qs HCl | | |
| 9D | 70 | 1.5 | 100 | .40 | 50% | PG | 0.1 M | — | 2.5 |
| | | | | | | | citric/ | | MTG |
| | | | | | | | qs HCl | | |
| 10D | 70 | 1.5 | 100 | .40 | 50% | PG | 0.1 M | — | 2.5 |
| | | | | | | | citric/ | | MTG |
| | | | | | | | qs HCl | | |
| 11D | 70 | 1.5 | 100 | .40 | 50% | PG | 0.1 M | — | — |
| | | | | | | | citric/ | | |
| | | | | | | | qs HCl | | |
| 12D | 70 | 1.5 | 100 | .40 | 50% | PG | 0.1 M | — | — |
| | | | | | | | citric/ | | |
| | | | | | | | qs HCl | | |
| 13D | 70 | 1.5 | 100 | .70 | 50% | PG | 0.1 M | — | 5 |
| | | | | | | | citric/ | | MTG |
| | | | | | | | qs HCl | | |
| 14D | 70 | 1.5 | 100 | .10 | 50% | PG | 0.1 M | — | 5 |
| | | | | | | | citric/ | | MTG |
| | | | | | | | qs HCl | | |
| 15D | 70 | 1.5 | 100 | .40 | 50% | PG | 0.05 M | — | 5 |
| | | | | | | | citric/ | | MTG |
| | | | | | | | qs HCl | | |
| 16D | 70 | 1.5 | 100 | .70 | 50% | PG | 0.025 M | — | 5 |
| | | | | | | | citric/ | | MTG |
| | | | | | | | qs HCl | | |
| 17D | 70 | 1.5 | 100 | .10 | 50% | PG | 0.025 M | — | 5 |
| | | | | | | | citric/ | | MTG |
| | | | | | | | qs HCl | | |

| | | % at 12 weeks and 50° C. | | | | |
|---|---|---|---|---|---|---|
| Experiment | | N-(n- | N-(n- | | % Equilibrium | |
| number | Head-space | propyl) | propyl) | | Mixture of | |
| and Group | Filler | isomer II | isomer I | | Isomers | % Impurities |
| 1A | N₂ | 12.33 | 0.48 | | 92.81 | 7.19 |
| 2A | N₂ | 12.09 | 7.88 | | 89.97 | 10.03 |
| 3A | N₂ | 11.95 | 6.13 | | 88.08 | 11.92 |
| 4A | N₂ | 12.78 | 6.44 | | 79.22 | 20.78 |
| 5A | N₂ | 13.07 | 5.50 | | 88.57 | 11.43 |
| 6A | N₂ | 10.71 | 6.71 | | 87.42 | 12.58 |
| 7A | N₂ | 11.81 | 0.34 | | 92.15 | 7.85 |
| 8A | N₂ | 11.80 | 4.27 | | 86.07 | 13.93 |
| 1B | N₂ | 10.90 | 9.50 | | 90.40 | 9.60 |
| 2B | N₂ | 9.10 | 3.20 | | 92.30 | 7.70 |
| 3B | N₂ | 10.70 | 1.40 | | 92.10 | 7.90 |
| 4B | N₂ | 9.50 | 3.80 | | 93.30 | 6.70 |
| 5B | N₂ | 10.90 | 9.70 | | 90.60 | 9.40 |
| 6B | N₂ | 9.30 | 4.40 | | 93.70 | 6.30 |
| 7B | N₂ | 9.70 | 9.40 | | 89.10 | 10.90 |
| 8B | N₂ | 9.90 | 2.70 | | 92.60 | 7.40 |
| 9B | N₂ | 9.50 | 3.10 | | 92.60 | 7.40 |
| 10B | N₂ | 9.90 | 1.70 | | 91.60 | 8.40 |
| 11B | N₂ | 10.20 | 2.20 | | 92.40 | 7.60 |
| 1C | N₂ | 10.10 | 3.40 | | 93.50 | 6.50 |
| 2C | N₂ | 10.00 | 4.30 | | 94.30 | 5.70 |
| 3C | N₂ | 10.40 | 9.60 | | 100.00 | — |
| 4C | N₂ | 9.90 | 3.00 | | 92.90 | 7.10 |
| 5C | N₂ | 9.90 | 3.00 | | 92.90 | 7.10 |
| 6C | N₂ | 8.40 | 8.20 | | 96.60 | 3.40 |
| 7C | N₂ | 8.40 | 8.80 | | 97.20 | 2.80 |
| 1D | air | 9.02 | 7.40 | | 96.42 | 3.58 |
| 2D | 5% O₂ | 9.03 | 7.40 | | 96.43 | 3.57 |
| 3D | air | 9.07 | 7.17 | | 96.24 | 3.76 |
| 5D | 10% O₂ | 9.14 | 7.38 | | 96.52 | 3.48 |
| 6D | 10% O₂ | 9.14 | 7.44 | | 96.58 | 3.42 |
| 7D | 5% O₂ | 9.20 | 7.16 | 8 | 96.36 | 3.64 |
| 8D | 1% O₂ | 9.20 | 7.14 | 8 | 96.34 | 3.66 |
| 9D | air | 9.19 | 7.17 | 8 | 96.36 | 3.64 |
| 10D | 5% O₂ | 9.24 | 7.14 | 8 | 96.38 | 3.62 |
| 11D | air | 9.18 | 6.94 | 8 | 96.12 | 3.88 |
| 12D | 5% O₂ | 9.18 | 6.92 | 8 | 96.10 | 3.90 |
| 13D | 10% O₂ | 9.18 | 6.96 | 8 | 96.14 | 3.86 |
| 14D | 10% O₂ | 9.20 | 7.05 | 8 | 96.25 | 3.75 |
| 15D | 10% O₂ | 9.21 | 7.44 | 8 | 96.65 | 3.35 |
| 16D | 10% O₂ | 9.11 | 7.51 | 8 | 96.62 | 3.38 |
| 17D | 10% O₂ | 9.11 | 6.79 | 8 | 95.90 | 4.10 |

| | | | Concentration | | | | |
|---|---|---|---|---|---|---|---|
| | | | of equilibrium | | | | |
| | | | mixture of | | | | |
| | | | N-(n-propyl) | | | | |
| | | | isomer I and | | | | |
| | | | N-(n-propyl) | | | | |
| Experiment | Equil. | | isomer II in | | Co-solvent | Acid | |
| number | temp | Equil. | pharmaceutical | | type and | concentration | Preser- |
| and Group | (°C) | time (hrs) | comp. (mg/mL) | ppH | amount | (M) | vative |
| 18D | 70 | 1.5 | 100 | 5.40 | 50% PG | 0.025 M | — |
| | | | | | | citric/ | |
| | | | | | | qs HCl | |
| 1E | | | 30 | 5.0 | 50% | qs citric | — |
| | | | | | glycerol | | |
| | | | | | formal | | |
| 2E | | | 30 | 5.0 | 50% N- | qs citric | — |
| | | | | | methyl 2- | | |
| | | | | | pyrrolidone | | |

| | | | % at 12 weeks and | | | |
|---|---|---|---|---|---|---|
| | | | 50° C. | | % | |
| Experiment | | | N-(n- | N-(n- | Equilibrium | |
| number | Antioxidant | Head-space | propyl) | propyl) | Mixture of | % |
| and Group | (mg/mL) | Filler | isomer II | isomer I | Isomers | Impurities |
| 18D | 5 MTG | 10% O₂ | 9.07 | 87.86 | 96.93 | 3.07 |
| 1E | — | air | | | 99.7 | |
| 2E | — | air | | | 94.9 | |

### EXAMPLE 4

Fifty-two liters of an injectable pharmaceutical composition containing 100 mg of equilibrium mixture of N-(n-propyl) isomer I and N-(n-propyl) isomer II per mL of composition were prepared as follows. 16.584 kg of Water for Injection (USP grade) sparged with nitrogen (NF grade) was added to a stainless steel compounding vessel and agitation was begun. Nitrogen was also used as an overlay to reduce oxygen exposure of the solution in the compounding vessel during manufacture. Approximately 1 kg of anhydrous citric acid (USP grade) was added to the water and the resulting mixture was agitated until the acid dissolved. 1.511 kg of a 10% (w/w) solution of hydrochloric acid (NF grade) in water (USP grade) was subsequently added to the mixture. 5.357 kg of a mixture containing approximately 97% of N-(n-propyl) isomer I and N-(n-propyl) isomer II (in a ratio of about 99:1) and 3% of one or more impurities was slowly added to the agitating mixture and was allowed to dissolve. The pH of the resulting solution was adjusted to 7.0±0.5 by adding 0.224 kg of a 10% (w/w) solution of hydrochloric acid in water. Equilibration of N-(n-propyl) isomer I and N-(n-propyl) isomer II was achieved by heating the solution to 70° C. ±10° C. for 105 minutes. Once equilibration was complete, as determined using HPLC, the solution was allowed to cool to 25° C. ±10° C., and 26.008 kg of propylene glycol (USP grade) was added to the agitating mixture. After the propylene glycol was completely mixed in, 0.26 kg of monothioglycerol (NF grade) was added to the solution, and the pH was readjusted to 5.4±0.3 by adding 2.349 kg of 10% (w/w) hydrochloric acid in water. The final volume was adjusted to 52.015 liters by adding 1.843 kg of water. The resulting composition contained 100 mg of the equilibrium mixture of N-(n-propyl) isomer I and N-(n-propyl) isomer II per mL of composition, 500 mg per mL of propylene glycol, citric acid at a concentration of 0.1 M, and monothioglycerol at a concentration of 5 mg/mL of composition.

The composition was filtered through a 6 micron pre-filter and then through a 0.2 micron final sterilizing filter, which was sterilized by moist-heat autoclaving for 60 minutes at 121° C. and tested for integrity using the pressure-hold method both prior to sterilization and after filtration. 20 mL flint type I serum glass vials (Wheaton Science Products, Millville, N.J.) were sterilized and depyrogenated in a dry heat tunnel at 250° C. for 240 minutes. 20 mm 4432/50 gray chlorobutyl siliconized stoppers (The West Company, Lionville, Pa.) were depyrogenated by washing and were sterilized by moist-heat autoclaving for 60 minutes at 121° C. Each of 2,525 vials was filled under sterile conditions with 20 mL of the resulting composition plus 0.6 mL overfill (20.6 mL/vial is 2.06 g/vial unit potency of pharmaceutical composition at 100 mg/mL of equilibrium mixture of N-(n-propyl) isomer I and N-(n-propyl) isomer II based on an actual drug substance lot potency of 97.1%), the vial head spaces were flushed with nitrogen, and the vials were sealed with the stoppers and overseals (20 mm aluminum seals, product # 5120-1125, The West Company, Lionville, Pa.).

### EXAMPLE 5

From about 0.125 mL to about 0.5 mL of a pharmaceutical composition having a pH of 5.4 and containing the equilibrium mixture of N-(n-propyl) isomer I and N-(n-propyl) isomer II present in an amount of 100 mg per mL of the pharmaceutical composition, where 100 mg per mL is the “potency-actual” number; citric acid present in an amount of 0.1 mmol per mL of the pharmaceutical composition; hydrochloric acid present in an amount of 19.58 mg of the concentrated acid (36-38% by weight potency) per mL of the pharmaceutical composition; sodium hydroxide present in an amount of 0.09 mg of a 1.0 M sodium hydroxide solution per mL of the pharmaceutical composition; sodium hydroxide present in an amount of 0.09 mg of a 10 M sodium hydroxide solution per mL of the pharmaceutical composition; propylene glycol present in an amount of 501.25 mg per mL of the pharmaceutical composition; and water present in an amount of 418.20 mg per mL of the pharmaceutical composition were administered to swine infected with Pasteurella multocida in order to determine its therapeutic efficacy. By Apotency-actual@ number, as used herein, is meant the actual mg per mL of the substantially pure mixture or equilibrium mixture of N-(n-propyl) isomer I and N-(n-propyl) isomer II present in the pharmaceutical composition.

Fifty clinically normal and healthy pigs having a consistent body weight of approximately 10 kg were selected from a pool of 60 animals. Selected animals (10 per treatment) were randomly assigned to treatment and sorted into pens accordingly. On day 0, each animal was inoculated endotracheally with 25 mL of Pasteurella multocida challenge culture. Each lot of animals was injected intramuscularly with a single dose of one of the following solutions approximately 1 hour post-inoculation: (1) about 1.5 mL of sterile 0.9% sodium chloride (saline); (2) about 0.5 mL of 25 mg/mL danofloxacin at a dose of 1.25 mg/kg of body weight; (3) about 0.125 mL of the pharmaceutical composition at a dose of 1.25 mg/kg of body weight; (4) about 0.25 mL the pharmaceutical composition at a dose of 2.5 mg/kg of body weight; or (5) about 0.5 mL of the pharmaceutical composition at a dose of 5 mg/kg of body weight. Only danofloxacin was re-administered on each of the following two days. All other treatments were given in a single dose injection. Temperatures and illness scores were recorded 6 hours post-challenge and once daily beginning at 24 hours post-challenge. Animals that developed severe pneumonia (i.e., illness score of 4) were euthanized and listed as a mortality. Animals that died during the course of the experiment were necropsied. Their lungs were removed and examined grossly for pneumonic lesions. An estimate of the percentage of affected lung tissue was determined and recorded. On day 5 post-challenge, all surviving animals were euthanized and necropsied as described above.

Assessment of efficacy was determined based upon comparison of mean daily illness scores, temperatures and lung-lesion scores. Differences between treatments for mean daily rectal temperatures and illness scores were evaluated by a repeated measures analysis of variance. Differences between mean lung-lesion scores for each treatment were evaluated using a factorial analysis of variance procedure. A comparison of mortality rates between treatments was performed using Chi-Square analysis and Fisher's Exact test.

The disease challenge in this study was relatively severe. Six hours post-challenge, the pigs were depressed, cyanotic and showed signs of dyspnea. Rectal temperatures were elevated in all treatment groups. The overall mortality rate for the study was 18% (9/50 pigs). Calculations of mean daily rectal temperatures indicated no statistically significant differences in these values among treatment groups. Although temperatures were elevated in all groups 6 hours post-challenge, the mean daily temperatures for all treatment groups remained within normal ranges. Animals treated with the pharmaceutical composition at 5 mg/kg of body weight or at 2.5 mg/kg of body weight, or with danofloxacin, displayed statistically significant (p<0.05) reductions in mean daily illness scores (about 2) compared to the mean daily illness scores of animals injected with saline (about 3). No significant differences were observed when comparing animals treated with three doses of danofloxacin to animals treated with a single dose of the pharmaceutical composition at 5 mg/kg of body weight or 2.5 mg/kg of body weight. Comparisons of the three treatments with the pharmaceutical composition indicated that pigs treated with the pharmaceutical composition at 5 mg/kg of body weight displayed statistically significant (p<0.05) lower clinical illness scores than pigs treated with the pharmaceutical composition at 1.25 mg/kg of body weight. No significant differences in illness scores were seen between pigs treated with the pharmaceutical composition at 5 mg/kg or the pharmaceutical composition at 2.5 mg/kg of body weight.

The effects of the various treatments upon mortality rates and lung-lesion scores are summarized in Table 3, below. Mortality rates ranged from 0% to 40% in the treated animals. Forty percent (4/10) of the animals in the saline control group died of pneumonia between 48-72 hours post-challenge. There was one death in the group treated with 2.5 mg/kg of body weight of the pharmaceutical composition and 4 deaths (40%) in the group treated with 1.25 mg/kg of body weight of the pharmaceutical composition. No deaths occurred in the groups treated with danofloxacin or 5 mg/kg of body weight of the pharmaceutical composition.

The mean lung-lesion score for the saline control pigs was 44%. Pigs treated with danofloxacin, or 2.5 mg/kg of body weight or 5 mg/kg of body weight of the pharmaceutical composition showed statistically significant (p<0.05) reductions in mean lung-lesion scores when compared to the saline controls. When comparing treated animals, animals treated with danofloxacin, or 2.5 mg/kg of body weight or 5 mg/kg of body weight of the pharmaceutical composition displayed statistically significant (p<0.05) reductions in mean lung lesion scores when compared to animals treated with 1.25 mg/kg of body weight of the pharmaceutical composition.

TABLE 3

| Treatment | | Mean Lung Lesion Score |
|---|---|---|
| (intramuscular injection) | Mortality | (%) |
| saline (1.5 ml) | 4/10 (40%) | 44.0 |
| Danofloxacin (1.25 mg/kg) | 0/10 (0%) | 1.9 |
| Pharmaceutical composition | 0/10 (0%) | 3.8 |
| (5 mg/kg) | | |
| Pharmaceutical composition | 1/10 (10%) | 6.6 |
| (2.5 mg/kg) | | |
| Pharmaceutical composition | 4/10 (40%) | 29.2 |
| (1.25 mg/kg) | | |

### EXAMPLE 6

From about 1.25 mL to about 5.0 mL of a pharmaceutical composition having a pH of 4.9 and containing a mixture of N-(n-propyl) isomer I and N-(n-propyl) isomer II in a ratio of from about 95% to about 99% of N-(n-propyl) isomer I and from about 1% to about 5% of N-(n-propyl) isomer II present in an amount of 200 mg per mL of the pharmaceutical composition, where 200 mg per mL is the “potency-actual” number; citric acid present in an amount of 85.09 mg per mL of the pharmaceutical composition; propylene glycol present in an amount of 253.40 mg per mL of the pharmaceutical composition; and water present in an amount of 541.46 mg per mL of the pharmaceutical composition were administered to calves with naturally occurring bacterial bovine respiratory disease.

Two hundred and thirteen calves (average weight of 200 kg) were purchased and co-mingled for approximately 2-3 days at the assembly point and transported approximately 1,000 miles by truck for delivery at a veterinary facility. No anti-bacterial treatment was given at any time during acquisition or pre-study handling. Upon arrival, the animals were unloaded into receiving pens, ear-tagged and provided with access to water and forage material. The animals were vaccinated with BOVISHIELD 4+L5 vaccine containing modified live viruses IBR, PI, BVD and BRSV, and a bacterin containing 5 servovars against Leptospira (Pfizer Animal Health). In addition, they were treated with the anti-parasitic agent DECTOMAX (Pfizer Animal Health) and implanted with a growth promotant (SYNOVEX-C, Syntex Laboratories). Beginning on the day after arrival, all animals were observed daily for clinical signs consistent with bovine respiratory disease. Individual animals exhibiting clinical signs of acute respiratory disease were selected (pulled) and their rectal temperatures were recorded. The selection criteria for inclusion in the study were a clinical presentation consistent with acute respiratory disease (i.e., illness score greater than or equal to 1 and less than 4) and pyrexia (rectal temperature greater than or equal to 104.0° F.). Once selected, animals were randomly allotted to one of five treatment groups using a randomized block allotment. All treatments were equally represented in each test pen (2 animals/treatment/pen). Each lot of animals was injected subcutaneously with a single dose of one of the following solutions on the day the selection criteria were met: (1) about 6.6 mL of sterile 0.9% sodium chloride (saline); (2) about 6.6 mL of MICOTIL 300 at a dose of 10 mg/kg of body weight; (3) about 1.25 mL of the pharmaceutical composition at a dose of 1.25 mg/kg of body weight; (4) about 2.5 mL of the pharmaceutical composition at a dose of 2.5 mg/kg of body weight; or (5) about 5 mL of the pharmaceutical composition at a dose of 5 mg/kg of body weight. All solutions were administered in a single-dose subcutaneous injection. During the post-treatment observation period, no further medication was administered. Temperatures and illness scores were recorded daily for all animals for 14 days post-treatment. Beginning 48 hours post-treatment, animals that exhibited an illness score of greater than or equal to 1 and a temperature of 104.0° F. were identified as re-pulls at the time of data analysis. Animals that developed severe pneumonia (i.e., illness score of 4) were euthanized and listed as a mortality. Animals that died during the course of the experiment were weighed and necropsied. Their lungs were removed and examined grossly for pneumonic lesions. An estimate of the percentage of affected lung tissue was determined and recorded. If possible, lung samples from a typically diseased area were collected from all animals for bacteriologic culture. On day 14, all surviving animals were euthanized. Animals were necropsied, and their lungs were assessed grossly for lesions as described above. Lung samples from all animals were collected for bacteriologic culture. The performance of the animals was assessed by evaluating individual weight gains. Each animal was weighed on days 7 and 14.

Assessment of efficacy was determined based upon analysis of mean daily illness scores, temperatures and lung-lesion scores. The proportion of successful responders in each treatment on day 14 was determined as the initial number of animals per treatment minus the number of mortalities and re-pulls. A comparison between treatment groups of the proportion of animals within each group exhibiting an illness score of 0 (normal) or greater than or equal to 1 on day 14 was evaluated using Chi-Square analysis and Fisher's Exact test. Differences in temperature and weight gain between treatments were evaluated using a repeated-measures ANOVA. The comparisons of mortality rates and responder rates between treatment groups were also performed using Chi-square analysis and Fisher's Exact test.

The outbreak of respiratory disease in this natural-disease study was extremely severe. The mortality rate for the saline controls was 75%. The mean lung-lesion score of the saline controls was 38.4%. The time course of the onset of clinical signs of disease was typical of that normally observed in a commercial feedyard with calves of this age and background. Calculations of mean daily rectal temperatures indicated statistically significant (p<0.01) reductions in mean daily rectal temperatures in all treatment groups when compared to the saline controls. Temperatures in the treated groups remained lower than those of the saline controls through day 7 of the study. Animals treated with either 2.5 mg/kg or 5 mg/kg of the pharmaceutical composition exhibited significantly (p<0.01) lower mean daily rectal temperatures than did animals treated with MICOTIL. Temperature responses of animals treated with 1.25 mg/kg of the pharmaceutical composition were similar to those of the MICOTIL controls. Therapeutic treatment of animals with either MICOTIL or the pharmaceutical composition at any dose level resulted in significant (p<0.01) reductions in mean daily illness scores compared to the saline controls. When comparing these treatments, calves treated with the pharmaceutical composition at 2.5 mg/kg displayed significantly (p<0.05) decreased mean daily illness scores compared to MICOTIL-treated calves. Calves treated with the pharmaceutical composition at either 1.25 mg/kg or 5 mg/kg displayed mean daily illness scores that were similar to calves treated with MICOTIL.

Re-pull rates, mortality and lung-lesion score data are summarized in Table 4, below. Seventy-five percent of the saline controls met the re-pull criteria in this study. Administration of MICOTIL or the pharmaceutical composition at 1.25 mg/kg resulted in reductions in the incidence of re-pulls (55% and 40%, respectively) relative to the saline controls. In contrast, re-pull rates for animals treated with either 2.5 mg/kg or 5 mg/kg of the pharmaceutical composition were significantly (p<0.01) lower than that of the saline controls. Re-pull rates for animals treated with 2.5 mg/kg of the pharmaceutical composition were significantly lower than those observed with MICOTIL. Re-pull rates for animals treated with either 1.25 mg/kg or 5 mg/kg of the pharmaceutical composition were reduced relative to MICOTIL. Fifteen of twenty (75%) saline control calves succumbed to pneumonia during the course of the study. Administration of MICOTIL resulted in a significant (p<0.01) reduction in the number of mortalities (25%) relative to the saline controls. Significant (p<0.01) reductions in mortality relative to the saline controls were also observed for all three groups of animals treated with the pharmaceutical composition. Comparative mortality rates were significantly (p<0.05) lower for animals treated with the pharmaceutical composition administered at 5 mg/kg relative to MICOTIL-treated calves. The two lower doses of the pharmaceutical composition provided reductions in mortality relative to MICOTIL. The mean lung-lesion score of the saline treated calves was 38.4%. Animals treated with either MICOTIL or the pharmaceutical composition at any dose level exhibited significant (p<0.01) reductions in mean lung-lesion scores relative to the saline controls. The pharmaceutical composition administered at either 2.5 mg/kg or 5 mg/kg provided reductions in mean lung-lesion scores relative to MICOTIL. Lung-lesion scores for animals treated with 1.25 mg/kg of the pharmaceutical composition were similar to those for animals treated with MICOTIL.

TABLE 4

| Treatment | | | |
|---|---|---|---|
| (subcutaneous | | | |
| injection) | Re-Pull Rate | Mortality Rate | Lung-Lesion Score |
| Saline | 15/20 (75%) | 15/20 (75%) | 38.4% |
| (6.6 mL) | | | |
| MICOTIL | 11/20 (55%) | 5/20 (25%) | 18.0% |
| (10 mg/kg) | | | |
| Pharmaceutical | 8/20 (40%) | 1/20 (5%) | 14.0% |
| Composition | | | |
| (1.25 mg/kg) | | | |
| Pharmaceutical | 2/20 (10%) | 1/20 (5%) | 8.6% |
| Composition | | | |
| (2.5 mg/kg) | | | |
| Pharmaceutical | 6/20 (30%) | 0/20 (0%) | 8.9% |
| Composition | | | |
| (5 mg/kg) | | | |

The proportion of responders for each treatment was calculated by subtracting the number of mortalities and re-pulls from the initial number of animals per treatment. Responder rates are summarized in Table 5. Twenty-five percent of the animals treated with MICOTIL met the responder criteria. Responder rates for animals treated with either 2.5 mg/kg or 5 mg/kg of the pharmaceutical composition were significantly (p<0.01 and p<0.05, respectively) improved relative to the MICOTIL treated animals. The responder rate for animals treated with 1.25 mg/kg of the pharmaceutical composition was greater than that observed for MICOTIL-treated animals. Clinically healthy calves were defined as those with an illness score of zero on day 14 (Table 5). In this study, only one of the saline controls was clinically healthy on day 14. Therapeutic administration of MICOTIL provided an increase in the number of healthy animals on day 14. The proportion of animals that were characterized as clinically healthy on day 14 in each of the treatments with the pharmaceutical composition was significantly (p<0.05) greater than the proportion in the saline control group. Similarly, the proportion of clinically healthy animals in all of the pharmaceutical composition treatment groups was greater than the proportion of clinically healthy animals in the MICOTIL group.

TABLE 5

| | | | Proportion of Clinically |
|---|---|---|---|
| Treatment | Responder Rates | | Healthy Animals |
| Saline | 3/20 | (15%) | 1/20 (5%) |
| (6.6 mL) | | | |
| MICOTIL | 5/20 | (25%) | 4/20 (20%) |
| (10 mg/kg) | | | |
| Pharmaceutical composition | 12/20 | (60) | 9/20 (45%) |
| (1.25 mg/kg) | | | |
| Pharmaceutical composition | 17/20 | (85%) | 8/20 (40%) |
| (2.5 mg/kg) | | | |
| Pharmaceutical composition | 14/20 | (70%) | 8/20 (40%) |
| (5 mg/kg) | | | |

Table 6, below, summarizes the effects of therapeutic treatment upon 7- and 14-day weight gains. Animals treated with either MICOTIL or with the pharmaceutical composition exhibited significantly (p<0.01) increased average daily gains at both days 7 and 14 relative to the saline controls. Animals treated with either 2.5 mg/kg or 5 mg/kg of the pharmaceutical composition exhibited improved weight gains relative to animals treated with MICOTIL. Animals treated with 1.25 mg/kg of the pharmaceutical composition exhibited similar weight gains to those treated with MICOTIL.

TABLE 6

| | Average Daily Weight Gain | Average Daily Weight Gain |
|---|---|---|
| Treatment | Over 7 Days (kg/day) | Over 14 Days (kg/day) |
| Saline | −1.18 | 0.36 |
| (6.6 mL) | | |
| MICOTIL | 0.60 | 0.78 |
| (10 mg/kg) | | |
| Pharmaceutical | 0.71 | 0.77 |
| composition | | |
| (1.25 mg/kg) | | |
| Pharmaceutical | 1.00 | 1.20 |
| composition | | |
| (2.5 mg/kg) | | |
| Pharmaceutical | 1.20 | 1.35 |
| composition | | |
| (5 mg/kg) | | |

### EXAMPLE 7

From about 1.25 mL to about 5 mL of a pharmaceutical composition having a pH of 6.0 and containing a mixture of N-(n-propyl) isomer I and N-(n-propyl) isomer II in a ratio of from about 95% to about 99% of N-(n-propyl) isomer I and from about 1% to about 5% of N-(n-propyl) isomer II present in an amount of 200 mg per mL of the pharmaceutical composition, where 200 mg per mL is the “potency-actual” number; citric acid present in an amount of 60.00 mg per mL of the pharmaceutical composition; propylene glycol present in an amount of 251.01 mg per mL of the pharmaceutical composition; and water present in an amount of 569.00 mg per mL of the pharmaceutical composition were administered to calves at a high risk for developing bacterial bovine respiratory disease.

Two hundred and twenty-two calves (average weight of 200 kg) were purchased, co-mingled for approximately 2 days at the assembly point and transported approximately 1,000 miles by truck for delivery at a veterinary facility. No anti-bacterial treatment was given at any time during acquisition or pre-study handling. Upon arrival, the animals were unloaded into receiving pens, ear-tagged and provided with access to water and forage material. All animals were vaccinated with BOVISHIELD 4+L5 vaccine containing modified live viruses IBR, PI, BVD and BRSV, and a bacterin containing 5 servovars against Leptospira (Pfizer Animal Health). In addition, they were treated with the anti-parasitic agent DECTOMAX (Pfizer Animal Health). On the day after arrival (day 0), the clinical condition of each animal was evaluated and an illness score recorded. On the day of allotment (day 0), animals that exhibited signs of fatigue, including mild depression or lack of rumen fill, in the absence of clinical signs of disease did not qualify for an illness score of greater than or equal to 1. Animals that exhibited an illness score of less than or equal to 1 and a body temperature of less than 104.0° F. were selected for inclusion in the study. Once selected, animals were randomly allotted to one of five treatment groups (20 calves per group) using a systematic randomized block allotment. The first ten animals selected were assigned to the first pen. Subsequent animals were assigned to pens in groups of ten until all pens were full. Each pen contained one or more animals form each treatment group. Each animals weight, body temperature and illness score were recorded prior to treatment on day 0. Each lot of animals was injected subcutaneously with a single dose of one of the following solutions within the first 30 hours after arrival: (1) about 6.6 mL of sterile 0.9% sodium chloride (saline); (2) about 6.6 mL of MICOTIL; (3) about 1.25 mL of the pharmaceutical composition at a dose of 1.25 mg/kg of body weight; (4) about 2.5 mL of the pharmaceutical composition at a dose of 2.5 mg/kg of body weight; or (5) about 5 mL of the pharmaceutical composition at a dose of 5 mg/kg of body weight. All solutions were administered in a single dose injection. Acute injection-site toleration observations were made at 24 and 48 hours post-injection. Temperatures and illness scores were recorded daily for all animals. Animals that exhibited an illness score of greater than or equal to 1 and a temperature of greater than or equal to 104° F. were identified as morbid (pulls) at the time of data analysis. Animals that developed severe pneumonia (i.e., illness score of 4) were euthanized and listed as a mortality. Animals that died during the course of the experiment were weighed and necropsied. Their lungs were removed and examined grossly for pneumonic lesions. An estimate of the percentage of affected lung tissue was determined and recorded. If possible, lung samples from a typically diseased area of the lung were collected from all animals for bacteriologic culture. On day 14, all surviving animals were euthanized and necropsied, and lung samples were assessed grossly for lesions and collected for bacteriologic culture as described above. The performance of the animals was assessed by evaluating individual weight gains. Each animal was weighed on days 7 and 14.

Assessment of efficacy was determined based upon comparison of mean daily illness scores, temperatures and lung-lesion scores. The proportion of successful responders in each treatment on day 14 was determined as the initial number of animals per treatment minus the number of mortalities and pulls. A comparison between treatment groups of the proportion of animals within each group exhibiting an illness score of 0 (normal) or greater than or equal to 1 on day 14 was evaluated using Chi-Square analysis and Fisher's Exact test. Differences in temperature and weight gain between treatments were evaluated by a repeated measures ANOVA. A comparison of mortality, morbidity and responder rates between treatments was also performed using Chi-Square analysis and Fisher's Exact test.

The outbreak of respiratory disease in this natural-disease study was moderately severe. The morbidity rate for the saline controls was 60%, and 25% of these animals died of acute pneumonia. The mean lung-lesion score of the saline controls was 24.3%. The time course of the onset of clinical signs of disease was typical of that normally observed in a commercial feedyard with calves of this age and background. Statistically significant (p<0.01) reductions in mean daily rectal temperatures were seen in all treatment groups when compared to the saline controls. Temperatures in the treated groups remained lower than those of the saline controls throughout day 10 of the study. Animals treated with any of the three doses of the pharmaceutical composition exhibited statistically significant (p<0.01) lower mean daily rectal temperatures than did animals treated with MICOTIL. The magnitude of the differences was greatest for animals treated with either 2.5 mg/kg or 5 mg/kg of the pharmaceutical composition. Metaphylactic treatment of calves with either MICOTIL or the pharmaceutical composition resulted in significant (p<0.01) reductions in mean daily illness scores compared to the saline controls. When comparing the antibiotic treatments, calves treated with 5 mg/kg of the pharmaceutical composition exhibited statistically significant (p<0.01) decreases in mean daily illness scores compared to animals treated with MICOTIL. Illness score responses of animals treated with either 1.25 mg/kg or 2.5 mg/kg of the pharmaceutical composition were similar to those of calves treated with MICOTIL.

Morbidity rates, mortality rates and lung-lesion score data are summarized in Table 7, below. In this moderately severe natural-infection study, the saline controls exhibited 60% morbidity. All antibiotic treatments exhibited significant (p<0.05) reductions in morbidity relative to the saline controls. Animals treated with the pharmaceutical composition exhibited numerical reductions in morbidity relative to the MICOTIL controls; however, none of the differences were statistically significant. Six of twenty (30%) saline control calves succumbed to bronchopneumonia during the course of the study. Administration of MICOTIL resulted in a reduction in the number of mortalities relative to the saline controls. Significant (p<0.05) reductions in mortality relative to the saline controls were observed for all three groups of animals treated with the pharmaceutical composition. The mean lung-lesion score for the saline control calves was 24.3%. Animals treated with MICOTIL or the pharmaceutical composition exhibited significant (p<0.01) reductions in mean lung-lesion scores relative to the saline controls. Calves treated with the pharmaceutical composition at 5 mg/kg exhibited significantly (p<0.05) lower lung-lesion scores than did animals treated with MICOTIL. Animals treated with either 1.25 mg/kg or 2.5 mg/kg of the pharmaceutical composition exhibited reductions in mean lung-lesion scores relative to calves treated with MICOTIL.

TABLE 7

| Treatment | Morbidity Rate | Mortality Rate | Lung-lesion Score |
|---|---|---|---|
| Saline | 12/20 (60%) | 6/20 (30%) | 24.3% |
| (6.6 mL) | | | |
| MICOTIL | 5/20 (25%) | 1/20 (5%) | 10.4% |
| (10 mg/kg) | | | |
| Pharmaceutical | 1/20 (5%) | 0/20 (0%) | 3.4% |
| composition | | | |
| (1.25 mg/kg) | | | |
| Pharmaceutical | 3/20 (15%) | 0/20 (0%) | 5.3% |
| composition | | | |
| (2.5 mg/kg) | | | |
| Pharmaceutical | 2/20 (10%) | 0/20 (0%) | 2.0% |
| composition | | | |
| (5 mg/kg) | | | |

The proportion of responders for each treatment was calculated by subtracting the number of mortalities and pulls from the initial number of animals per treatment. Responder rates are summarized in Table 8. Differences in the relative responder rates observed for the various treatments were similar to differences described above for morbidity rates. Clinically healthy calves were defined as those with an illness score of zero on day 14. In this study, only one of the saline controls was clinically healthy on day 14. A significantly (p<0.01) greater proportion of the animals treated with either MICOTIL or the pharmaceutical composition were observed to be clinically healthy on day 14 relative to the saline controls. Similarly, a greater proportion of the animals treated with any of the doses of the pharmaceutical composition were determined to be more clinically healthy than of those treated with MICOTIL. However, these differences were not statistically significant (p>0.05).

TABLE 8

| | | Proportion of Clinically |
|---|---|---|
| Treatment | Responder Rates | Healthy Animals |
| Saline | 8/20 (40%) | 1/20 (5%) |
| (6.6 ml) | | |
| MICOTIL | 15/20 (75%) | 10/20 (50%) |
| (10 mg/kg) | | |
| Pharmaceutical composition | 19/20 (95%) | 14/20 (70%) |
| (1.25 mg/kg) | | |
| Pharmaceutical composition | 17/20 (85%) | 13/20 (65%) |
| (2.5 mg/kg) | | |
| Pharmaceutical composition | 18/20 (90%) | 14/20 (70%) |
| (5 mg/kg) | | |

Table 9 summarizes the effects of metaphylactic treatment upon 7- and 14-day weight gains. Animals treated with either MICOTIL or the pharmaceutical composition exhibited significantly (p<0.05) increased average daily gains at both days 7 and 14 relative to the saline controls. Weight-gain responses for the various antibiotic treatments were similar.

TABLE 9

| | Average Daily | Average Daily |
|---|---|---|
| | Weight Gain Over | Weight Gain Over |
| Treatment | 7 Days (kg/day) | 14 Days (kg/day) |
| Saline | 0.21 | 0.46 |
| (6.6 mL) | | |
| MICOTIL | 1.15 | 0.94 |
| (10 mg/kg) | | |
| Pharmaceutical | 1.09 | 1.20 |
| composition | | |
| (1.25 mg/kg) | | |
| Pharmaceutical | 0.96 | 1.00 |
| composition | | |
| (2.5 mg/kg) | | |
| Pharmaceutical composition | 1.55 | 1.25 |
| (5 mg/kg) | | |

Acute injection sites were examined at 24 and 48 hours and assessments were made using the following scale: O-no affected area (swelling/inflammation) observed; 1=small-affected area (swelling/inflammation) less than 6 inches in diameter; 2=medium-affected area (swelling/inflammation) 6-8 inches in diameter; 3=large-affected area (swelling/inflammation) greater than 8 inches in diameter; 4=extreme-affected area (swelling/inflammation) greater than 8 inches and/or radiating into the brisket or causing lameness. Grades were assigned depending upon the size and extent of the acute affected area. The 24 and 48 hour assessments are summarized in Table 10. In this study, the statistical significance of differences in the proportion of animals within each treatment scoring of greater than or equal to 2 at 24 hours post-injection was evaluated. There were no statistically significant differences between treatments. However, the number of abnormal injection sites was greater for animals treated with MICOTIL than for animals treated with the pharmaceutical composition.

TABLE 10

| | 24 hr assessment | | | | | 48 hr assessment | | | | |
|---|---|---|---|---|---|---|---|---|---|---|
| Treatment | 0 | 1 | 2 | 3 | 4 | 0 | 1 | 2 | 3 | 4 |
| Saline | 100% | 0% | 0% | 0% | 0% | 100% | 0% | 0% | 0% | 0% |
| (6.6 mL) | | | | | | | | | | |
| MICOTIL | 80% | 15% | 5% | 0% | 0% | 95% | 5% | 0% | 0% | 0% |
| (10 mg/kg) | | | | | | | | | | |
| Pharmaceutical | 100% | 0% | 0% | 0% | 0% | 100% | 0% | 0% | 0% | 0% |
| Composition | | | | | | | | | | |
| (1.25 mg/kg) | | | | | | | | | | |
| Pharmaceutical | 100% | 0% | 0% | 0% | 0% | 100% | 0% | 0% | 0% | 0% |
| composition | | | | | | | | | | |
| (2.5 mg/kg) | | | | | | | | | | |
| Pharmaceutical | 100% | 0% | 0% | 0% | 0% | 100% | 0% | 0% | 0% | 0% |
| composition | | | | | | | | | | |
| (5 mg/kg) | | | | | | | | | | |

### EXAMPLE 8

From about 0.5 mL to about 2 mL of a pharmaceutical composition having a pH of 6.1 and containing a mixture of N-(n-propyl) isomer I and N-(n-propyl) isomer II in a ratio of from about 95% to about 99% of N-(n-propyl) isomer I and from about 1% to about 5% of N-(n-propyl) isomer II present in an amount of 50 mg per mL of the pharmaceutical composition, where 50 mg per mL is the “potency-actual” number; citric acid present in an amount of 15.00 mg per mL of the pharmaceutical composition; propylene glycol present in an amount of 250.13 mg per mL of the pharmaceutical composition; and water present in an amount of 734.43 mg per mL of the pharmaceutical composition were administered to pigs at a high risk for developing an Actinobacillus pleuropneumoniae infection.

One hundred and thirty clinically healthy pigs having an average body weight of approximately 10 kg were purchased, identified with an ear tag and acclimated to the study site 2 days before the study began. On day −1, all animals were weighed and 100 animals were selected for consistency of body weight (about 10 kg) and lack of signs of clinical abnormalities. Selected animals (20 per treatment) were randomly assigned to treatment and sorted into individual pens. A group of 25 additional animals were randomly assigned as seeder pigs (5 per treatment). On day 0, animals were injected intramuscularly with a single dose of one of the following solutions: (1) about 1.5 mL of sterile 0.9% sodium chloride (saline); (2) about 0.5 mL of 25 mg/mL danofloxacin at a dose of 1.25 mg/kg of body weight; (3) about 0.5 mL of the pharmaceutical composition at a dose of 2.5 mg/kg of body weight; (4) about 1 mL the pharmaceutical composition at a dose of 5 mg/kg of body weight; or (5) about 2 mL of the pharmaceutical composition at a dose of 10 mg/kg of body weight. Only danofloxacin was re-administered on each of the following two days. All other treatments were administered in a single dose injection. Concurrently, on day 0, the 25 seeder pigs were challenged with 3 mL/nare of Actinobacillus pleuropneumonia challenge culture. Five infected seeder animals were added to each pen of 20 test animals. Test animals and seeder pigs were co-mingled. Seeder pigs that died during the study were removed from the pens. At 48 hours post-challenge, surviving seeder pigs were removed from treatment pens and euthanized. Temperatures and illness scores were recorded daily. Animals that died during the course of the experiment were necropsied. The lungs were removed and examined grossly for pneumonic lesions. An estimate of the percentage of affected lung tissue was determined and recorded. Animals with a lung-lesion score of greater than or equal to 5% were considered morbid. On day 7, all surviving animals were euthanized. Animals were necropsied and lungs removed and examined grossly for pneumonic lesions.

Assessment of efficacy was determined based upon comparison of mean daily illness scores, temperatures and lung-lesion scores. Differences between treatments for mean daily rectal temperatures and illness scores were evaluated by a repeated-measures analysis of variance. A comparison between treatment groups of the proportion of animals within each group exhibiting an illness score of 0 (normal) or greater than or equal to 1 on day 7 were evaluated using Chi-Square analysis and Fishers Exact test. Comparisons of morbidity (greater than or equal to 5% lung-lesion score) and mortality rates between treatment groups were performed using Chi-Square analysis and Fisher's Exact test.

Eighty percent of the seeder pigs died of pneumonia within 24 hours of challenge, indicating adequate exposure of the test animals to the bacterial pathogen. Temperatures in the saline-treated pigs began to rise on day 1 post-exposure and remained significantly elevated throughout the duration of the study compared to the pharmaceutical composition- and danofloxacin-treated groups. Mean daily rectal temperatures for the pharmaceutical composition-treated groups at 5 mg/kg and 10 mg/kg were significantly (p<0.05) lower than the danofloxacin-treated pigs. Initial reductions in temperature occurred in pigs treated with the pharmaceutical composition at 2.5 mg/kg compared to pigs treated with danofloxacin. However, differences in mean daily rectal temperatures between these two treatments were not statistically significant (p>0.05). Statistically significant (p<0.05) elevations in mean daily illness scores were seen in the saline-treated pigs when compared to danofloxacin- and pharmaceutical composition-treated animals. However, there were no differences in mean daily illness scores between the danofloxacin- and pharmaceutical composition-treated pigs. A comparison between treatment groups of the proportion of animals within each group exhibiting an illness score of 0 (normal) or greater than or equal to 1 on day 7 showed no differences among any of the treated groups.

Data summarizing the morbidity and mortality rates are presented in Table 11. Morbidity criteria were established from pigs having a mean lung-lesion score of greater than or equal to 5%. A statistically significant (p<0.05) increase in morbidity rate was seen in the saline control group in this study compared to the danofloxacin- and pharmaceutical composition-treated pigs. However, there were no differences in morbidity rates between the danofloxacin- and pharmaceutical composition-treated pigs.

TABLE 11

| Treatment | Proportion of Morbid Pigs |
|---|---|
| Saline (1.5 mL) | 13/20 (65%) |
| Danofloxacin (1.25 mg/kg) | 6/20 (30%) |
| Pharmaceutical composition (2.5 mg/kg) | 6/20 (30%) |
| Pharmaceutical composition (5 mg/kg) | 1/20 (5%) |
| Pharmaceutical composition (10 mg/kg) | 5/20 (25%) |

The effects of the various treatments upon mortality rates and lung-lesion scores are summarized in Table 12, below. The mean lung-lesion score for the saline control pigs was 22.2%. Pigs treated with danofloxacin and the pharmaceutical composition showed statistically significant (p<0.05) reductions in mean lung-lesion scores when compared to the saline controls. However, there were no statistically significant (p<0.05) differences in mean lung-lesion scores between the danofloxacin- and pharmaceutical compositon-treated pigs.

TABLE 12

| Treatment | Mortality | Mean Lung-lesion Score |
|---|---|---|
| Saline (1.5 mL) | 2/20 (10%) | 22.2% |
| Danofloxacin (1.25 mg/kg) | 0/20 (0%) | 4.8% |
| Pharmaceutical composition | 0/20 (0%) | 4.6% |
| (2.5 mg/kg) | | |
| Pharmaceutical composition | 0/20 (0%) | 0.6% |
| (5 mg/kg) | | |
| Pharmaceutical composition | 0/20 (0%) | 3.1% |
| (10 mg/kg) | | |

### EXAMPLE 9

From about 3 mL to about 6 mL of a pharmaceutical composition having a pH of 5.4 and containing an equilibrium mixture of N-(n-propyl) isomer I and N-(n-propyl) isomer II present in an amount of 100 mg per mL of the pharmaceutical composition, where 100 mg per mL is the potency-actual number; citric acid present in an amount of 0.1 mmol per mL of the pharmaceutical composition; hydrochloric acid present in an amount of 19.58 mg of the concentrated acid (36-38% by weight potency) per mL of the pharmaceutical composition; sodium hydroxide present in an amount of 0.09 mg of a 1.0 M sodium hydroxide solution per mL of the pharmaceutical composition; sodium hydroxide present in an amount of 0.09 mg of a 10 M sodium hydroxide solution per mL of the pharmaceutical composition; propylene glycol present in an amount of 501.25 mg per mL of the pharmaceutical composition; and water present in an amount of 418.20 mg per mL of the pharmaceutical composition were administered to calves challenged with 2 mL of a coccidia challenge culture containing 125,000 sporulated oocysts with a species percent count of 93% Eimeria bovis, 4% Eimeria aubumenis and 3% Eimeria zuernii coccidia oocysts.

Sixty naive calves weighing approximately 110-125 kg were purchased from local dairies, weighed, identified by ear tag, and observed for general health assessments. Animals considered physically abnormal, undersized or moribund on arrival were excluded from the study. Calves were housed in five holding pens (12 animals/pen). Calves were held for 7 days prior to challenge in order to acclimate them to the facility. Prior to challenge, calves were excluded from the study at the discretion of the investigator. On days −6, −4 and −2 pre-challenge, fecal samples were obtained for semi-quantitative oocyst counts. On day −4 pre-challenge, oocysts, if present, were speciated.

On day 8 post-arrival (study day 0), calves were inoculated orally with the Eimeria culture. Beginning on day 1, temperatures were determined and recorded at approximately the same time each day for the duration of the study. Attitude, hydration and fecal consistency scores were evaluated daily. Post-challenge, fecal samples were collected on days 2, 4, 6, 8 and 10. Oocysts were speciated on day 10 post-challenge. On day 10 post-challenge, fifty animals were randomly allotted to one of five treatment groups using a randomized block allotment. Treatments were equally represented in each pen. Animals were injected subcutaneously with a single dose of one of the following solutions: (1) about 4 mL of sterile 0.9% sodium chloride (saline); (2) about 4 mL of 300 mg/mL MICOTIL at a dose of 10 mg/kg of body weight; (3) about 6 mL of the pharmaceutical composition at a dose of 5 mg/kg of body weight; (4) about 3 mL of the pharmaceutical composition at a dose of 2.5 mg/kg of body weight; or animals were drenched dosed orally with (5) about 2 oz. of amprolium in a 9.6% oral solution at a dose of 10 mg/kg of body weight. Only amprolium was re-administered on each of the following four days. All other treatments were given in a single dose injection. Fecal samples were examined semi-quantitatively post-treatment for shedding of coccidia oocysts on days 12, 14, 16 and 18. Beginning on day 19 and continuing through day 28, daily fecal samples were evaluated for semi-quantitative counts. Speciation of shed oocysts was performed on days 19-21, 23, 26 and 28. Calves that died during the course of the study or that were euthanized due to a moribund condition associated with clinical coccidiosis were counted as mortalities. Mortalities were necropsied and gross findings were recorded. At the termination of the study on day 28, all remaining animals were weighed, euthanized and examined post-mortem.

Assessment of drug efficacy was determined based upon analysis of mean daily clinical scores, temperature and oocyst shedding. Differences in clinical scores and temperature between different treatments were evaluated by repeated measures ANOVA. Differences in weight gain were determined by factorial ANOVA. Comparisons of mortality rates and oocyst shedding between treatments was performed using Chi-Square analysis and Fisher's Exact test.

At day 19 post-challenge, oocyst shedding was detected. Mean daily rectal temperatures for each treatment remained in the normal range during the duration of the study. No significant differences (p>0.05) between treatment groups were detected. Clinical score assessments included scores for fecal consistency, hydration and attitude. Attitude and fecal scores indicated that calves treated with MICOTIL, amprolium or the pharmaceutical composition at either dose level responded favorably to treatment, compared to the saline control calves. Increases in fecal scores, hydration scores and attitude scores corresponded to the time of detectable shedding of oocysts (day 19). Animals treated with amprolium, MICOTIL, or the pharmaceutical composition at either dose level displayed statistically significant reductions (p<0.05) in mean daily fecal consistency scores compared to the saline treated calves. The increased fecal scores occurred 2-3 days prior to shedding of oocysts and remained elevated throughout the 28-day study. No differences were detected upon comparison of calves treated with amprolium, MICOTIL or pharmaceutical composition. Calves treated with amprolium displayed statistically significant reductions (p<0.05) in mean daily hydration scores compared to the saline treated calves. No differences in hydration scores were seen between calves treated with amprolium, MICOTIL or pharmaceutical composition. Treatment of calves with amprolium, MICOTIL or the pharmaceutical composition at either dose level resulted in significant reductions (p<0.05) in mean daily attitude scores compared to the saline controls. The differences in attitude scores were noted between the amprolium and saline treated calves at the time of peak oocyst shedding. Animals treated with MICOTIL or the pharmaceutical composition at either dose level exhibited numerical reductions in attitude scores relative to the saline control calves during the last seven days of the study. No significant differences (p>0.05) were seen between the MICOTIL, amprolium or pharmaceutical composition treatment groups.

Mortality rates are summarized in Table 13. Five calves died due to coccidiosis in this study. Three calves died on day 23 post-challenge and two calves died on day 28 post-infection. Two animals died in each of the saline and MICOTIL treatment groups. One animal in the amprolium treated group died during the course of the study. There were no mortalities among animals treated with the pharmaceutical composition. There were no statistically significant (p>0.05) differences in mortality rates among the non-saline-treated animals.

TABLE 13

| Treatment | Mortality |
|---|---|
| Saline (6 mL) | 2/10 (20%) |
| Amprolium (2 oz.) | 1/10 (10%) |
| MICOTIL (10 mg/kg) | 2/10 (20%) |
| Pharmaceutical composition | 0/10 (0%) |
| (2.5 mg/kg) | |
| Pharmaceutical composition | 0/10 (0%) |
| (5 mg/kg) | |

Table 14 summarizes the effects of treatments upon weight gains. Positive average daily gains were seen in all treatment groups. Increases in weight gain were seen in calves treated with the pharmaceutical composition and with amprolium compared to animals in the saline and MICOTIL treatment groups. MICOTIL- and saline-treated animals responded similarly when assessing the 21-day average daily gains. However, no statistical differences in weight gain were seen among the non-saline-treated groups.

TABLE 14

| | 21-day Average Daily Weight Gain |
|---|---|
| Treatment | (kg) |
| Saline (6 mL) | 0.30 |
| Amprolium (2 oz.) | 0.60 |
| MICOTIL (10 mg/kg) | 0.21 |
| Pharmaceutical composition | 0.45 |
| (2.5 mg/kg) | |
| Pharmaceutical composition | 0.44 |
| (5 mg/kg) | |

Eimeria oocyst shedding was monitored prior to challenge and post-challenge. Oocyst shedding was first detectable on day 19 post-challenge. In this study, statistically significant (p<0.05) increases in oocyst shedding were seen in saline-treated animals when compared to the MICOTIL-, amprolium- and pharmaceutical composition-treated animals. Also, MICOTIL-treated animals displayed statistically significant (p<0.05) increases oocyst shedding compared to animals treated with amprolium. However, no statistically significant (p>0.05) differences in oocyst shedding were seen when comparing the MICOTIL- and amprolium-treated calves to calves treated with either dose of the pharmaceutical composition.

In this study, 40-100% of the animals in the saline control group were consistently shedding oocysts on days 19, 20, 21, 23, 26 and 28 post-challenge. Animals treated with amprolium, MICOTIL or the pharmaceutical composition displayed decreased oocyst shedding compared to the saline controls. In this study, E. bovis accounted for approximately 60-100% of the shed oocysts per sample. E. aubumenis and E. zuernii accounted for approximately 10-40% of the shed oocysts per sample. There was an apparent increase in the shedding of E. zuernii oocysts on day 28 post-challenge, which corresponded to a decrease in shedding of E. bovis oocysts. However, over the entire monitored shedding period, none of the compounds tested appeared to significantly alter the speciation profiles of the shed oocysts.

At necropsy, the majority of the animals displayed gross pathology consistent with a moderate to severe coccidial infection. In this study, calves from all treatment groups showed signs of hemorrhagic ilietis and colitis. Fourteen percent of the calves in this study (7/50) displayed no gross pathology at necropsy. However, the shed oocysts from calves in each of the treatment groups suggested some level of coccidia infection in these animals.

The present invention is not to be limited in scope by the specific embodiments disclosed in the Examples, which are intended as illustrations of a few aspects of the invention. Any embodiments which are functionally equivalent are within the scope of this invention. Indeed, various modifications of the invention in addition to those shown and described herein will become apparent to those skilled in the art and are intended to fall within the appended claims.

All references disclosed herein are hereby incorporated by reference in their entirety.

What is claimed is:

1. A method for treating or preventing a bacterial or protozoal infection in a mammal, comprising administering to a mammal in need of such treatment or prevention a single dose of an effective amount of a composition comprising:
(a) mixture of:
a compound of formula (I): 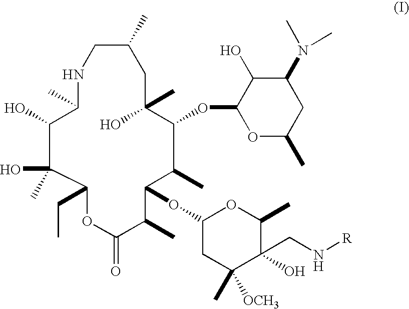
or a or a pharmaceutically acceptable salt thereof, and
a compound of formula (II): 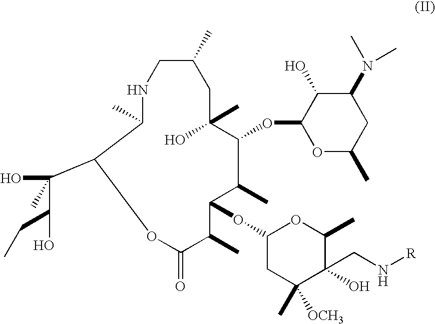
or a pharmaceutically acceptable salt thereof,
wherein both R groups are identical and are selected from the group consisting of hydrogen, a C₁-C₁₀ straight or branched chain alkyl group, and a C₃-C₇ cycloalkyl group; and
(b) a pharmaceutically acceptable vehicle.

2. The method of claim 1, wherein the compound of formula I, or a pharmaceutically acceptable salt thereof, and the compound of formula II, or a pharmaceutically acceptable salt thereof, are present in a ratio of about 90%±4% to about 10%±4%, respectively.

3. The method of claim 1 or 2, wherein R is n-propyl.

4. The method of claim 1 or 2, wherein the pharmaceutically acceptable vehicle comprises:
(a) water;
(b) one or more acids present at a total concentration of from about 0.2 mmol to about 1.0 mmol per mL of the mixture; and
(c) one or more water-miscible co-solvents present in an amount of from about 250 to about 750 mg per mL of the composition.

5. The method of claim 4, wherein the one or more water-miscible co-solvents are selected from the group consisting of ethanol, isopropanol, diethylene glycol monomethyl ether, diethylene glycol butyl ether, diethylene glycol monoethyl ether, diethylene glycol dibutyl ether, polyethylene glycol-300, polyethylene glycol-400, propylene glycol, glycerine, 2-pyrrolidone, N-methyl 2-pyrrolidone, glycerol formal, dimethyl sulfoxide, dibutyl sebecate, polysorbate 80, and mixtures thereof.

6. The method of claim 5, wherein the one or more water-miscible co-solvents is propylene glycol.

7. The method of claim 6, wherein propylene glycol is present in an amount of from about 450 to about 550 mg per mL of the composition.

8. The method of claim 4, wherein the composition further comprises one or more antioxidants present in an amount of from about 0.01 mg to about 10 mg per mL of the composition.

9. The method of claim 8, wherein the one or more antioxidants is selected from the group consisting of sodium bisulfite, sodium sulfite, sodium metabisulfite, sodium thiosulfate, sodium formaldehyde sulfoxylate, 1-ascorbic acid, erythorbic acid, acetylcysteine, cysteine, monothioglycerol, thioglycollic acid, thiolactic acid, thiourea, dithiothreitol, dithioerythreitol, glutathione, ascorbyl palmitate, butylated hydroxyanisole, butylated hydroxytoluene, nordihydroguaiaretic acid, propyl gallate, α-tocopherol, and mixtures thereof.

10. The method of claim 9, wherein the one or more antioxidants is monothioglycerol.

11. The method of claim 10, wherein monothioglycerol is pr-sent in an amount of from about 4 mg to about 6 mg per mL of the composition.

12. The method of claim 4, wherein the composition further comprises one or more preservatives present in an amount of from about 0.01 to about 10 mg per mL of the composition.

13. The method of claim 12, wherein the one or more preservatives is selected from the group consisting of benzalkonium chloride, benzethonium chloride, benzoic acid, benzyl alcohol, methylparaben, ethylparaben, propylparaben, butylparaben, sodium benzoate, phenol, and mixtures thereof.

14. The method of claim 13, wherein the one or more preservatives is phenol and is present in an amount of from about 2.0 to about 3.0 mg per mL of the composition.

15. The method of claim 4, wherein the one or more acids are selected from the group consisting of acetic acid, benzenesulfonic acid, citric acid, hydrobromic acid, hydrochloric acid, D- and L-lactic acid, methanesulfonic acid, phosphoric acid, succinic acid, sulfuric acid, D- and L-tartaric acid, p-toluenesulfonic acid, adipic acid, aspartic acid, camphorsulfonic acid, 1,2-ethanedisulfonic acid, laurylsulfuric acid, glucoheptonic acid, gluconic acid, 3-hydroxy-2-naphthoic acid, 1-hydroxy-2-naphthoic acid, 2-hydroxyethanesulfonic acid, malic acid, mucic acid, nitric acid, naphthalenesulfonic acid, palmitic acid, D-glucaric acid, stearic acid, maleic acid, malonic acid, fumaric acid, benzoic acid, cholic acid, ethanesulfonic acid, glucuronic acid, glutamic acid, hippuric acid, lactobionic acid, lysinic acid, mandelic acid, napadisylic acid, nicotinic acid, polygalacturonic acid, salicylic acid, sulfosalicylic acid, tryptophanic acid, and mixtures thereof.

16. The method of claim 1 or 2, wherein the bacterial or protozoal infection is selected from the group consisting of bovine respiratory disease, swine respiratory disease, bovine infectious keratoconjunctivitis, bovine coccidiosis, porcine ileitis, bovine mastitis, calf enteric disease, porcine enteric disease, canine pneumonia, feline pneumonia, canine pyoderma, feline pyoderma, pasteurellosis, anaplasmosis, infectious keratinitis; pneumonia, otitis media, sinusitus, bronchitis, tonsillitis, and mastoiditis associated with infection by Streptococcus pneumoniae, Haemophilus influenzae, Moraxella catarrhalis, Staphylococcus aureus, or Peptostreptococcus spp.; pharynigitis, rheumatic fever, and glomerulonephritis associated with infection by Streptococcus pyogenes, Groups C and G streptococci, Clostridium diptheriae, or Actinobacillus haemolyticum; respiratory tract infections associated with infection by Mycoplasma pneumoniae, Legionella pneumophila, Streptococcus pneumoniae, Haemophilus influenzae, or Chlamydia pneumoniae; uncomplicated skin and soft tissue infections, abscesses, osteomyelitis, and puerperal fever associated with infection by Staphylococcus aureus, coagulase-positive staphylococci (i.e., S. epidermidis, S. hemolyticus, etc.), Streptococcus pyogenes, Streptococcus agalactiae, Streptococcal groups C-F (minute-colony streptococci), viridans streptococci, Corynebacterium minutissimum, Clostridium spp., or Bartonella henselae; uncomplicated acute urinary tract infections associated with infection by Staphylococcus saprophyticus or Enterococcus spp.; urethritis and cervicitis; sexually transmitted diseases associated with infection by Chlamydia trachomatis, Haemophilus ducreyi, Treponema pallidum, Ureaplasma urealyticum, or Neiserria gonorrheae; toxin diseases associated with infection by S. aureus, or Groups A, B, and C streptococci; ulcers associated with infection by Helicobacter pylon; systemic febrile syndromes associated with infection by Borrelia recurrentis; Lyme disease associated with infection by Borrelia burgdorferi; conjunctivitis, keratitis, and dacrocystitis associated with infection by Chlamydia trachomatis, Neisseria gonorrhoeae, S. aureus, S. pneumoniae, S. pyogenes, H. influenzae, or Listeria spp.; disseminated Mycobacterium avium complex (MAC) disease associated with infection by Mycobacterium avium, or Mycobacterium intracellulare; gastroenteritis associated with infection by Campylobacter jejuni; intestinal protozoa associated with infection by Cryptosporidium spp.; odontogenic infection associated with infection by viridans streptococci; persistent cough associated with infection by Bordetella pertussis; gas gangrene associated with infection by Clostridium perfringens or Bacteroides spp.; atherosclerosis associated with infection by Helicobacter pylori or Chlamydia pneumoniae; cow footrot associated with infection by Fusobacterium spp.; cow metritis associated with infection by E. coli; cow hairy warts associated with infection by Fusobacterium necrophorum or Bacteroides nodosus; cow premature abortion associated with infection by protozoa; urinary tract infection in dogs and cats associated with infection by E. coli; skin and soft tissue infections in dogs and cats associated with infection by Staph. epidermidis, Staph. intermedius, coagulase neg. Staph. or P. multocida; dental or mouth infections in dogs and cats associated with infection by Alcaligenes spp., Bacteroides spp., Clostridium spp., Enterobacter spp., Eubacterium, Peptostreptococcus, Porphyromonas, or Prevotella; and infections of horses associated with Actinobacillus equi, Rodococcus equi, Streptococcus equi, and Streptococcus zooepidemicus.

17. A method for increasing acute or chronic injection-site toleration in a mammal, comprising administering to a mammal in need thereof a single dose of an effective amount of a composition comprising:
(a) a mixture of:
a compound of formula (I): 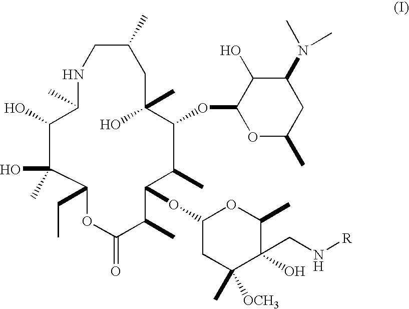
or a pharmaceutically acceptable salt thereof and
a compound of formula (II): 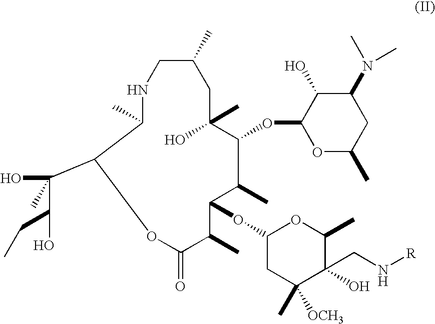
or a pharmaceutically acceptable salt thereof, wherein both R groups are identical and are selected from the group consisting of hydrogen, a C₁-C₁₀ straight or branched chain alkyl group, and a C₃-C₇ cycloalkyl group; and
(b) a pharmaceutically acceptable vehicle.

18. The method of claim 17, wherein the compound of formula I, or a pharmaceutically acceptable salt thereof, and the compound of formula II, or a pharmaceutically acceptable salt thereof, are present in a ratio of about 90%±4% to about 10%±4%, respectively.

19. The method of claim 17 or 18, wherein R is n-propyl.

20. The method of claim 17 or 18, wherein the pharmaceutically acceptable vehicle comprises:
(a) water;
(b) one or more acids present at a total concentration of from about 0.2 mmol to about 1.0 mmol per mL of the mixture; and
(c) one or more water-miscible co-solvents present in an amount of from about 250 to about 750 mg per mL of the composition.

21. The method of claim 20, wherein the one or more water-miscible co-solvents are selected from the group consisting of ethanol, isopropanol, diethylene glycol monomethyl ether, diethylene glycol butyl ether, diethylene glycol monoethyl ether, diethylene glycol dibutyl ether, polyethylene glycol-300, polyethylene glycol-400, propylene glycol, glycerine, 2-pyrrolidone, N-methyl 2-pyrrolidone, glycerol formal, dimethyl sulfoxide, dibutyl sebecate, polysorbate 80, and mixtures thereof.

22. The method of claim 21, wherein the one or more water-miscible co-solvents is propylene glycol.

23. The method of claim 22, wherein propylene glycol is present in an amount of from about 450 to about 550 mg per mL of the composition.

24. The method of claim 20, wherein the composition further comprises one or more antioxidants present in an amount of from about 0.01 mg to about 10 mg per mL of the composition.

25. The method of claim 24, wherein the one or more antioxidants is selected from the group consisting of sodium bisulfite, sodium sulfite, sodium metabisulfite, sodium thiosulfate, sodium formaldehyde sulfoxylate, 1-ascorbic acid, erythorbic acid, acetylcysteine, cysteine, monothioglycerol, thioglycollic acid, thiolactic acid, thiourea, dithiothreitol, dithioerythreitol, glutathione, ascorbyl palmitate, butylated hydroxyanisole, butylated hydroxytoluene, nordihydroguaiaretic acid, propyl gallate, □-tocopherol, and mixtures thereof.

26. The method of claim 25, wherein the one or more antioxidants is monothioglycerol.

27. The method of claim 26, wherein monothioglycerol is present in an amount of from about 4 mg to about 6 mg per mL of the composition.

28. The method of claim 20, wherein the composition further comprises one or more preservatives present in an amount of from about 0.01 to about 10 mg per mL of the composition.

29. The method of claim 28, wherein the one or more preservatives is selected from the group consisting of benzalkonium chloride, benzethonium chloride, benzoic acid, benzyl alcohol, methylparaben, ethylparaben, propylparaben, butylparaben, sodium benzoate, phenol, and mixtures thereof.

30. The method of claim 29, wherein the one or more preservatives is phenol and is present in an amount of from about 2.0 to about 3.0 mg per mL of the composition.

31. The method of claim 20, wherein the one or more acids are selected from the group consisting of acetic acid, benzenesulfonic acid, citric acid, hydrobromic acid, hydrochloric acid, D- and L-lactic acid, methanesulfonic acid, phosphoric acid, succinic acid, sulfuric acid, D- and L-tartaric acid, p-toluenesulfonic acid, adipic acid, aspartic acid, camphorsulfonic acid, 1,2-ethanedisulfonic acid, laurylsulfuric acid, glucoheptonic acid, gluconic acid, 3-hydroxy-2-naphthoic acid, 1-hydroxy-2-naphthoic acid, 2-hydroxyethanesulfonic acid, malic acid, mucic acid, nitric acid, naphthalenesulfonic acid, palmitic acid, D-glucaric acid, stearic acid, maleic acid, malonic acid, fumaric acid, benzoic acid, cholic acid, ethanesulfonic acid, glucuronic acid, glutamic acid, hippuric acid, lactobionic acid, lysinic acid, mandelic acid, napadisylic acid, nicotinic acid, polygalacturonic acid, salicylic acid, sulfosalicylic acid, tryptophanic acid, and mixtures thereof.

32. The method of claim 17 or 18, wherein the bacterial or protozoal infection is selected from the group consisting of bovine respiratory disease, swine respiratory disease, bovine infectious keratoconjunctivitis, bovine coccidiosis, porcine ileitis, bovine mastitis, calf enteric disease, porcine enteric disease, canine pneumonia, feline pneumonia, canine pyoderma, feline pyoderma, pasteurellosis, anaplasmosis, infectious keratinitis; pneumonia, otitis media, sinusitus, bronchitis, tonsillitis, and mastoiditis associated with infection by Streptococcus pneumoniae, Haemophilus influenzae, Moraxella catarrhalis, Staphylococcus aureus, or Peptostreptococcus spp.; pharynigitis, rheumatic fever, and glomerulonephritis associated with infection by Streptococcus pyogenes, Groups C and G streptococci, Clostridium diptheriae, or Actinobacillus haemolyticum; respiratory tract infections associated with infection by Mycoplasma pneumoniae, Legionella pneumophila, Streptococcus pneumoniae, Haemophilus influenzae, or Chlamydia pneumoniae; uncomplicated skin and soft tissue infections, abscesses, osteomyelitis, and puerperal fever associated with infection by Staphylococcus aureus, coagulase-positive staphylococci (i.e., S. epidermidis, S. hemolyticus, etc.), Streptococcus pyogenes, Streptococcus agalactiae, Streptococcal groups C—F (minute-colony streptococci), viridans streptococci, Corynebacterium minutissimum, Clostridium spp., or Bartonella henselae; uncomplicated acute urinary tract infections associated with infection by Staphylococcus saprophyticus or Enterococcus spp.; urethritis and cervicitis; sexually transmitted diseases associated with infection by Chlamydia trachomatis, Haemophilus ducreyi, Treponema pallidum, Ureaplasma urealyticum, or Neiserria gonorrheae; toxin diseases associated with infection by S. aureus, or Groups A, B, and C streptococci; ulcers associated with infection by Helicobacter pylori; systemic febrile syndromes associated with infection by Borrelia recurrentis; Lyme disease associated with infection by Borrelia burgdorferi; conjunctivitis, keratitis, and dacrocystitis associated with infection by Chlamydia trachomatis, Neisseria gonorrhoeae, S. aureus, S. pneumoniae, S. pyogenes, H. influenzae, or Listeria spp.; disseminated Mycobacterium avium complex (MAC) disease associated with infection by Mycobacterium avium, or Mycobacterium intracellulare;
gastroenteritis associated with infection by Campylobacter jejuni; intestinal protozoa associated with infection by Cryptosporidium spp.; odontogenic infection associated with infection by viridans streptococci; persistent cough associated with infection by Bordetella pertussis; gas gangrene associated with infection by Clostridium perfringens or Bacteroides spp.; atherosclerosis associated with infection by Helicobacter pylori or Chlamydia pneumoniae; cow footrot associated with infection by Fusobacterium spp.; cow metritis associated with infection by E. coli; cow hairy warts associated with infection by Fusobacterium necrophorum or Bacteroides nodosus; cow premature abortion associated with infection by protozoa; urinary tract infection in dogs and cats associated with infection by E. coli; skin and soft tissue infections in dogs and cats associated with infection by Staph. epidermidis, Staph. intermedius, coagulase neg. Staph. or P. multocida; dental or mouth infections in dogs and cats associated with infection by Alcaligenes spp., Bacteroides spp., Clostridium spp., Enterobacter spp., Eubacterium, Peptostreptococcus, Porphyromonas, or Prevotella; and infections of horses associated with Actinobacillus equi, Rodococcus equi, Streptococcus equi, and Streptococcus zooepidemicus.

33. A combination comprising:
(a) a composition comprising:
(1) a mixture of:
a compound of formula (I): 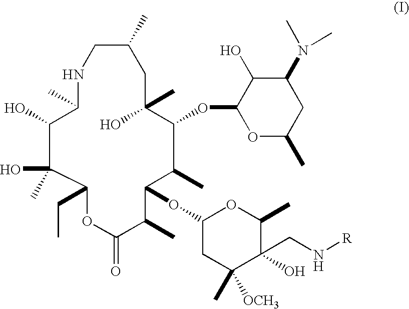
or a pharmaceutically acceptable salt thereof, wherein both R groups are identical and are selected from the group consisting of hydrogen, a C₁-C₁₀ straight or branched chain alkyl group, and a C₃-C₇ cycloalkyl group; and
(2) a pharmaceutically acceptable vehicle; and
(b) instructions for use in a single-dose administration.

34. The combination of claim 33, wherein the compound of formula I, or a pharmaceutically acceptable salt thereof, and the compound of formula II, or a pharmaceutically acceptable salt thereof, are present in a ratio of about 90%±4% to about 10%±4%, respectively.

35. The combination of claim 33 or 34, wherein R is n-propyl.

36. The combination of claim 33 or 34, wherein the pharmaceutically acceptable vehicle comprises:
(a) water;
(b) one or more acids present at a total concentration of from about 0.2 mmol to about 1.0 mmol per mL of the mixture; and
(c) one or more water-miscible co-solvents present in an amount of from about 250 to about 750 mg per mL of the composition.

37. The combination of claim 36, wherein the one or more water-miscible co-solvents are selected from the group consisting of ethanol, isopropanol, diethylene glycol monomethyl ether, diethylene glycol butyl ether, diethylene glycol monoethyl ether, diethylene glycol dibutyl ether, polyethylene glycol-300, polyethylene glycol-400, propylene glycol, glycerine, 2-pyrrolidone, N-methyl 2-pyrrolidone, glycerol formal, dimethyl sulfoxide, dibutyl sebecate, polysorbate 80, and mixtures thereof.

38. The combination of claim 37, wherein the one or more water-miscible co-solvents is propylene glycol.

39. The combination of claim 38, wherein propylene glycol is present in an amount of from about 450 to about 550 mg per mL of the composition.

40. The combination of claim 39, wherein the composition further comprises one or more antioxidants present in an amount of from about 0.01 mg to about 10 mg per mL of the composition.

41. The combination of claim 40, wherein the one or more antioxidants is selected from the group consisting of sodium bisulfite, sodium sulfite, sodium metabisulfite, sodium thiosulfate, sodium formaldehyde sulfoxylate, 1-ascorbic acid, erythorbic acid, acetylcysteine, cysteine, monothioglycerol, thioglycollic acid, thiolactic acid, thiourea, dithiothreitol, dithioerythreitol, glutathione, ascorbyl palmitate, butylated hydroxyanisole, butylated hydroxytoluene, nordihydroguaiaretic acid, propyl gallate, α-tocopherol, and mixtures thereof.

42. The combination of claim 41, wherein the one or more antioxidants is monothioglycerol.

43. The combination of claim 42, wherein monothioglycerol is present in an amount of from about 4 mg to about 6 mg per mL of the composition.

44. The combination of claim 36, wherein the composition further comprises one or more preservatives present in an amount of from about 0.01 to about 10 mg per mL of the composition.

45. The combination of claim 44, wherein the one or more preservatives is selected from the group consisting of benzalkonium chloride, benzethonium chloride, benzoic acid, benzyl alcohol, methylparaben, ethylparaben, propylparaben, butylparaben, sodium benzoate, phenol, and mixtures thereof.

46. The combination of claim 45, wherein the one or more preservatives is phenol and is present in an amount of from about 2.0 to about 3.0 mg per mL of the composition.

47. The combination of claim 36, wherein the one or more acids are selected from the group consisting of acetic acid, benzenesulfonic acid, citric acid, hydrobromic acid, hydrochloric acid, D- and L-lactic acid, methanesulfonic acid, phosphoric acid, succinic acid, sulfuric acid, D- and L-tartaric acid, p-toluenesulfonic acid, adipic acid, aspartic acid, camphorsulfonic acid, 1,2-ethanedisulfonic acid, laurylsulfuric acid, glucoheptonic acid, gluconic acid, 3-hydroxy-2-naphthoic acid, 1-hydroxy-2-naphthoic acid, 2-hydroxyethanesulfonic acid, malic acid, mucic acid, nitric acid, naphthalenesulfonic acid, palmitic acid, D-glucaric acid, stearic acid, maleic acid, malonic acid, fumaric acid, benzoic acid, cholic acid, ethanesulfonic acid, glucuronic acid, glutamic acid, hippuric acid, lactobionic acid, lysinic acid, mandelic acid, napadisylic acid, nicotinic acid, polygalacturonic acid, salicylic acid, sulfosalicylic acid, tryptophanic acid, and mixtures thereof.

48. The method of claim 1 wherein the single dose is in a range of from about 0.5 mg of compounds of formulae 1 and 2 taken together per kg of body weight (mg/kg) to about 20 mg/kg.

49. The method of claim 48 wherein the single dose is in a range of from about 1.25 mg of compounds of formulae 1 and 2 taken together per kg of body weight (mg/kg) to about 10 mg/kg.

50. The method of claim 49 wherein the single dose is in a range of from about 2.0 mg compounds of formulae 1 and 2 taken together per kg of body weight (mg/kg) to about 5.0 mg/kg.
